(19) United States
(12) Patent Application Publication
(10) Pub. No.: US 2024/0287525 A1
(43) Pub. Date: Aug. 29, 2024

(54) COMPOSITIONS AND METHODS FOR TREATING CANCER

(71) Applicants: OSLO UNIVERSITETSSYKEHUS HF, Oslo (NO); UNIVERSITETET I OSLO, Oslo (NO)

(72) Inventors: Fahri SAATCIOGLU, Oslo (NO); Omer Faruk KUZU, Oslo (NO); Nora PALLMANN, Oslo (NO); Yang JIN, Oslo (NO)

(21) Appl. No.: 18/570,687

(22) PCT Filed: Jun. 17, 2022
(86) PCT No.: PCT/IB2022/000348

§ 371 Date: Dec. 15, 2023

Related U.S. Application Data
(60) Provisional application No. 63/211,896, filed on Jun. 17, 2021.

(51) Int. Cl. C12N 15/113

(52) U.S. Cl. CPC C12N 15/1137; C12Y 105/01015; C12N 2310/14

(57) ABSTRACT

The present invention relates to the use of oligonucleotides to treat cancer, and in particular to treat prostate cancer.

### CROSS-REFERENCE TO RELATED APPLICATION

The present application claims priority to U.S. Provisional Patent Application No. 63/211,896, filed Jun. 17, 2021, which is hereby incorporated by reference in its entirety.

### FIELD OF THE INVENTION

The present invention relates to the use of oligonucleotides to treat cancer, and in particular to treat prostate cancer.

### BACKGROUND OF THE INVENTION

According to current estimates, more than 34,000 Americans will die from prostate cancer (PCa) in 2021 (American Cancer Society, 2021). PCa is the most common cancer in men and the number two killer overall. Treatment options for PCa include active surveillance/watchful waiting (AS/WW), radiation therapy, brachy therapy, prostatectomy, androgen deprivation therapy, immunotherapy, and chemotherapy according to the 2010 and 2019 National Comprehensive Cancer Network (NCCN) guidelines Prostate Cancer, Version 2.2019, NCCN Clinical Practice Guidelines in Oncology (Mohler et al., J Natl Compr Canc Netw. 2019 May 1; 17(5):479-505). In advanced disease, in patients with metastatic PCa that fail earlier therapies, the prognosis is rather poor, with a median life expectancy of 13 months (e.g., Aly et al., Scand. J. of Urology, 2020; 54(2): 115-121)

Accordingly, improved methods for treating PCa are desperately needed. The present invention addresses this urgent need.

### SUMMARY OF THE INVENTION

One carbon (1C) metabolism has a key role in metabolic programming with both mitochondrial (m1C) and cytoplasmic (c1C) components. Here we show that Activating Transcription Factor 4 (ATF4) exclusively activates gene expression involved in m1C, but not c1C cycle in prostate cancer (PCa) cells. This includes activation of methylenetetrahydrofolate dehydrogenase 2 (MTHFD2) expression, the central player in the m1C cycle. Consistent with the key role of m1C cycle in PCa, MTHFD2 knockdown inhibited PCa cell growth, prostatosphere formation and growth of patient-derived xenograft (PDX) organoids. In addition, therapeutic silencing of MTHFD2 by systemically administered nanoliposomal siRNA profoundly inhibited tumor growth in multiple preclinical PCa mouse models. Consistently, MTHFD2 expression is significantly increased in human PCa and a gene expression signature based on the m1C cycle has significant prognostic value. Furthermore, MTHFD2 expression is coordinately regulated by ATF4 and the oncoprotein c-MYC, which has been implicated in PCa. These data support that the m1C cycle is essential for PCa progression and thus serves as a biomarker and therapeutic target.

Accordingly, provided herein are compositions and methods for treating cancer (e.g., prostate cancer) by blocking MTHFD2 expression and/or activity.

For example, certain embodiments provide an oligonucleotide composition comprising one or more nucleotides, or salts thereof, or a pharmaceutical agent that induces the production of the one or more oligonucleotide, wherein the one or more oligonucleotides is at least 80% (e.g., at least 80%, 85%, 90%, 91%, 92%, 93%, 94%, 95%, 96%, 97%, 98%, 99%, or 100%) identical to any one of SEQ ID NOs: 1 to 63 and 126 to 140 or the complement thereof, wherein the oligonucleotides hybridize to the MTHFD2 of SEQ ID NO:64 or an mRNA encoded thereof. In some embodiments, the oligonucleotide is an RNA (e.g., siRNA). In some embodiments, the siRNA comprises a sense strand and an antisense strand, wherein the antisense strand hybridizes to an mRNA encoded by SEQ ID NO:64.

In some embodiments, the one or more oligonucleotides is a modified oligonucleotide. The present invention is not limited to particular oligonucleotide compositions. Exemplary modifications are described herein.

In preferred embodiments, the one or more oligonucleotides is present in a pharmaceutical composition (e.g., comprising one or several lipids).

Further embodiments provide an expression vector encoding an siRNA (e.g., an shRNA) comprising a nucleic acid that expresses an RNA selected from SEQ ID NOs: 1-63 or 126 to 140 linked to a nucleic acid encoding the complement of an RNA selected from SEQ ID NOs: 1-63 or 126 to 140.

In some preferred embodiments, the one or more oligonucleotides or a vector encoding the oligonucleotides is administered systemically or locally (e.g., to the prostate or other site of a tumor or cancer) to a subject with cancer (e.g., prostate cancer). In some embodiments, administration of the one or more oligonucleotides results in down-regulation of expression of the MTHFD2 gene in the tissue of a subject. In some embodiments, the administration of the one or more nucleotides ameliorates one or more symptoms of cancer in the subject.

Further embodiments provide a method of treating cancer, comprising: administering a nucleic acid (e.g., those described herein) to a subject diagnosed with cancer (e.g., prostate cancer).

Yet other embodiments provide the use of nucleic acid (e.g., those described herein) to treat cancer (e.g., prostate cancer) in a subject.

In some preferred embodiments, the present invention provides a double-stranded ribonucleic acid (dsRNA) agent for inhibiting expression of methylenetetrahydrofolate dehydrogenase 2 (MTHFD2), wherein said dsRNA agent comprises a sense strand and an antisense strand forming a double-stranded region, wherein said antisense strand comprises at least 15 contiguous nucleotides and excluding any overhang shares at least 80% identity to a sequence selected from the group consisting of SEQ ID NOs: 1 to 63 or 126 to 140. In some preferred embodiments, the present invention provides a double-stranded ribonucleic acid (dsRNA) agent for inhibiting expression of methylenetetrahydrofolate dehydrogenase 2 (MTHFD2), wherein said dsRNA agent comprises a sense strand and an antisense strand forming a double-stranded region, wherein said antisense strand comprises at least 15 contiguous nucleotides and differs, preferably excluding any overhang, from a sequence selected from the group consisting of SEQ ID NOs: 1 to 63 or 126 to 140 by no more than 5 nucleotides. In some preferred embodiments, the antisense strand differs, preferably excluding any overhang, from a sequence selected from the group consisting of SEQ ID NOs: 1 to 63 or 126 to 140 by no more than 4 nucleotides. In some preferred embodiments, the antisense strand differs, preferably excluding any overhang, from a sequence selected from the group consisting of SEQ ID NOs: 1 to 63 or 126 to 140 by no more than 3 nucleotides. In some preferred embodiments, the antisense strand differs, preferably excluding any overhang, from a sequence selected from the group consisting of SEQ ID NOs: 1 to 63 or 126 to 140 by no more than 2 nucleotides. In some preferred embodiments, the antisense strand differs, preferably excluding any overhang, from a sequence selected from the group consisting of SEQ ID NOs: 1 to 63 or 126 to 140 by no more than 1 nucleotide. In some preferred embodiments, the antisense strand differs, preferably excluding any overhang, from a sequence selected from the group consisting of SEQ ID NOs: 1 to 63 or 126 to 140 by 0) nucleotides.

In some preferred embodiments, the dsRNA agent comprises at least one modified nucleotide. In some preferred embodiments, the at least one of said modified nucleotides is selected from the group consisting of a 2′-O-methyl modified nucleotide, a 2′-fluoro modified nucleotide, a 2′-deoxy thymidine (dT) nucleotide, a nucleotide comprising a 5′ phosphorothioate group, and a terminal nucleotide linked to a cholesteryl derivative or a dodecanoic acid bisdecylamide group.

In some preferred embodiments, each strand is no more than 30 nucleotides in length. In some preferred embodiments, each strand is independently 17-25 nucleotides in length. In some preferred embodiments, each strand is independently 19-25 nucleotides in length. In some preferred embodiments, each strand is independently 19-23 nucleotides in length.

In some preferred embodiments, at least one strand comprises a 3′ overhang of at least 1 nucleotide. In some preferred embodiments, at least one strand comprises a 3′ overhang of at least 2 nucleotides.

In some preferred embodiments, the dsRNA agent further comprises at least one phosphorothioate or methylphosphonate internucleotide linkage.

In some preferred embodiments, the present invention provides a cell containing the dsRNA agent as described above. In some preferred embodiments, the present invention provides an expression vector encoding the dsRNA agent as described above. In some preferred embodiments, the present invention provides a pharmaceutical composition for inhibiting expression of a MTHFD2 gene comprising the dsRNA agent as described above.

In some preferred embodiments, the present invention provides methods of inhibiting MTHFD2 expression in a cell, the method comprising: (a) contacting the cell with the dsRNA agent or a pharmaceutical composition as described above; and (b) maintaining the cell produced in step (a) for a time sufficient to obtain degradation of the mRNA transcript of an gene, thereby inhibiting expression of the gene in the cell. In some preferred embodiments, the cell is within a subject.

In some preferred embodiments, the present invention provides methods of treating a subject having a disorder that would benefit from reduction in MTHFD2 expression, comprising administering to the subject a therapeutically effective amount of the dsRNA agent or pharmaceutical composition as described above, thereby treating said subject. In some preferred embodiments, the disorder is prostate cancer.

In some preferred embodiments, the present invention provides methods of inhibiting the expression of MTHFD2 in a subject, the method comprising administering to said subject a therapeutically effective amount of the dsRNA agent or a pharmaceutical composition as described above, thereby inhibiting the expression of MTHFD2 in said subject.

In some preferred embodiments, the present invention provides a dsRNA agent or pharmaceutical composition as described above for use in treating a subject having a disorder that would benefit from reduction in MTHFD2 expression. In some preferred embodiments, the disorder is prostate cancer.

In some preferred embodiments, the present invention provides methods of inhibiting MTHFD2 expression in a subject in need thereof comprising: administering to the subject a dsRNA agent comprising a sense strand and an antisense strand forming a double stranded region, wherein said antisense strand comprises at least 15 contiguous nucleotides and excluding any overhang is at least 80% identical to a selected portion of the nucleotide sequence of the complement of nucleotides 1 to 1100 of SEQ ID NO:64. In some preferred embodiments, the antisense strand differs, preferably excluding any overhang, from the selected portion of SEQ ID NO:64 by no more than 4 nucleotides. In some preferred embodiments, the antisense strand differs, preferably excluding any overhang, from the selected portion of SEQ ID NO:64 by no more than 3 nucleotides. In some preferred embodiments, the antisense strand differs, preferably excluding any overhang, from the selected portion of SEQ ID NO:64 by no more than 2 nucleotides. In some preferred embodiments, the antisense strand differs, preferably excluding any overhang, from the selected portion of SEQ ID NO:64 by no more than 1 nucleotide. In some preferred embodiments, the antisense strand differs, preferably excluding any overhang, from the selected portion of SEQ ID NO:64 by 0 nucleotides.

In some preferred embodiments, the dsRNA agent comprises at least one modified nucleotide. In some preferred embodiments, the at least one of said modified nucleotides is selected from the group consisting of a 2′-O-methyl modified nucleotide, a 2′-fluoro modified nucleotide, a 2′-deoxy thymidine (dT) nucleotide, a nucleotide comprising a 5′-phosphorothioate group, and a terminal nucleotide linked to a cholesteryl derivative or a dodecanoic acid bisdecylamide group.

In some preferred embodiments, each strand is no more than 30 nucleotides in length. In some preferred embodiments, each strand is independently 17-25 nucleotides in length. In some preferred embodiments, each strand is independently 19-25 nucleotides in length. In some preferred embodiments, each strand is independently 19-23 nucleotides in length.

In some preferred embodiments, at least one strand comprises a 3′ overhang of at least 1 nucleotide. In some preferred embodiments, at least one strand comprises a 3′ overhang of at least 2 nucleotides.

In some preferred embodiments, the dsRNA agent further comprises at least one phosphorothioate or methylphosphonate internucleotide linkage.

In some preferred embodiments, the dsRNA agent is formulated with a pharmaceutically acceptable carrier.

In some preferred embodiments, the subject has cancer. In some preferred embodiments, the cancer is prostate cancer.

In some preferred embodiments, the present invention provides an iRNA agent for inhibiting expression of methylenetetrahydrofolate dehydrogenase 2 (MTHFD2), wherein said iRNA agent comprises an antisense strand comprising at least 15 contiguous nucleotides and excluding any overhang shares at least 80% identity to a sequence selected from the group consisting of SEQ ID NOs: 1 to 63 or 126 to 140. In some preferred embodiments, the antisense strand differs, preferably excluding any overhang, from a sequence selected from the group consisting of SEQ ID NOs: 1 to 63 or 126 to 140 by no more than 5 nucleotides. In some preferred embodiments, the antisense strand differs, preferably excluding any overhang, from a sequence selected from the group consisting of SEQ ID NOs: 1 to 63 or 126 to 140 by no more than 4 nucleotides. In some preferred embodiments, the antisense strand differs, preferably excluding any overhang, from a sequence selected from the group consisting of SEQ ID NOs: 1 to 63 or 126 to 140 by no more than 3 nucleotides. In some preferred embodiments, the antisense strand differs, preferably excluding any overhang, from a sequence selected from the group consisting of SEQ ID NOs: 1 to 63 or 126 to 140 by no more than 2 nucleotides. In some preferred embodiments, the antisense strand differs, preferably excluding any overhang, from a sequence selected from the group consisting of SEQ ID NOs: 1 to 63 or 126 to 140 by no more than 1 nucleotide. In some preferred embodiments, the antisense strand differs, preferably excluding any overhang, from a sequence selected from the group consisting of SEQ ID NOs: 1 to 63 or 126 to 140 by 0 nucleotides.

In some preferred embodiments, the iRNA agent comprises at least one modified nucleotide. In some preferred embodiments, the at least one of said modified nucleotides is selected from the group consisting of a 2′-O-methyl modified nucleotide, a 2′-fluoro modified nucleotide, a 2′-deoxy thymidine (dT) nucleotide, a nucleotide comprising a 5′-phosphorothioate group, and a terminal nucleotide linked to a cholesteryl derivative or a dodecanoic acid bisdecylamide group.

In some preferred embodiments, the antisense strand is no more than 30 nucleotides in length. In some preferred embodiments, the antisense strand is independently 17-25 nucleotides in length. In some preferred embodiments, the antisense strand is independently 19-25 nucleotides in length. In some preferred embodiments, the antisense strand is independently 19-23 nucleotides in length. In some preferred embodiments, the antisense strand comprises a 3′ overhang of at least 1 nucleotide. In some preferred embodiments, the antisense strand comprises a 3′ overhang of at least 2 nucleotides.

In some preferred embodiments, the iRNA agent further comprises at least one phosphorothioate or methylphosphonate internucleotide linkage.

In some preferred embodiments, the iRNA agent is a dsRNA agent.

In some preferred embodiments, the present invention provides a cell containing the iRNA agent as described above.

In some preferred embodiments, the present invention provides a pharmaceutical composition for inhibiting expression of a MTHFD2 gene comprising the iRNA agent as described above.

In some preferred embodiments, the present invention provides methods of inhibiting MTHFD2 expression in a cell, the method comprising: (a) contacting the cell with the iRNA agent or a pharmaceutical composition as described above; and (b) maintaining the cell produced in step (a) for a time sufficient to obtain degradation of the mRNA transcript of an gene, thereby inhibiting expression of the gene in the cell. In some preferred embodiments, the cell is within a subject.

In some preferred embodiments, the present invention provides methods of treating a subject having a disorder that would benefit from reduction in MTHFD2 expression, comprising administering to the subject a therapeutically effective amount of the iRNA agent or a pharmaceutical composition as described above, thereby treating said subject. In some preferred embodiments, the disorder is prostate cancer.

In some preferred embodiments, the present invention provides methods of inhibiting the expression of MTHFD2 in a subject, the method comprising administering to said subject a therapeutically effective amount of the iRNA agent or a pharmaceutical composition as described above, thereby inhibiting the expression of MTHFD2 in said subject.

In some preferred embodiments, the present invention provides an iRNA agent or pharmaceutical composition as described above for use in treating a subject having a disorder that would benefit from reduction in MTHFD2 expression. In some preferred embodiments, the disorder is prostate cancer.

Additional embodiments are described herein.

### BRIEF DESCRIPTION OF THE DRAWINGS

FIG. 1: MTHFD2 knockdown inhibits growth of PCa cells, PDX-derived organoids, and tumor xenografts. A. MTHFD2 knockdown efficiency was determined in LNCaP and DU145 cells that were transfected with either scrambled siRNA or two independent MTHFD2-specific siRNAs for 48 hours by both qPCR and Western analyses. B. LNCaP, VCaP, or 22Rv1 cells transfected with control or MTHFD2-specific siRNAs were cultured for the indicated times, and cell numbers were determined by trypan blue staining. *p<0.01. MTHFD2 knockdown hinders colony (C-D) and prostatosphere (E) formation ability of PCa cells. F. Viability of normal prostate cells is not affected by MTHFD2 knockdown. G. MTHFD2 expression and knockdown efficiency in various LuCaP organoids were assessed by Western analysis. H. MTHFD2 knockdown significantly decreased LuCaP organoid formation without affecting cell death, in LuCaP 35 and LuCaP 96 that express MTHFD2, but not in LuCaP 136, which does not express MTHFD2. I. Nanoliposomal systemic delivery of MTHFD2-specific siRNA profoundly inhibited the growth of VCaP and 22Rv1 xenograft tumors in vivo.

FIG. 2: MTHFD2 expression is increased in PCa. A. Basal levels of MTHFD2 mRNA and protein were determined in various PCa cell lines and the normal prostate cell line by qPCR and Western analysis, respectively. B. MTHFD2 expression was analyzed by immunohistochemistry in matched benign prostate and PCa specimens from 24 patients. Representative images and quantification of staining is shown. C. Tissue microarrays with normal prostate (n=223) and primary prostate tumors (n=860) were analyzed by immunohistochemistry. Middle panel shows increased MTHFD2 expression with increasing Gleason grade of the samples. Representative images and quantification of staining intensity are shown. D. In a subset of the samples used for IHC analysis, the correlation of ATF4 and MTHFD2 staining scores is depicted. r denotes Pearson correlation between the two stainings.

FIG. 3: Mitochondrial IC gene expression is deregulated in PCa and a gene expression signature derived from it is strongly associated with PCa prognosis. A-B. Expression of MTHFD2 and SHMT2 is upregulated in primary and/or metastatic PCa compared to benign samples. N: Normal, P: Primary, M: Metastatic. C. m1C but not c1C cycle genes are upregulated in primary PCa samples in the TCGA dataset. D. A gene expression signature consisting of the three ATF4-regulated m1C cycle genes (MTHFD2+MTHFD1L+SHMT2) is significantly associated with recurrence-free survival in the TCGA dataset.

FIG. 4: A. LNCaP cells were transfected with MTHFD2 (siMTHFD2 #1) or non-targeting control siRNAs. Cells were cultured for the indicated time points, collected, and MTHFD2 mRNA levels were determined by qRT-PCR. Black bar shows expression of MTHFD2 in non-transfected cells. B. LNCaP and DU145 cells were reverse transfected with either control siRNA or two independent siRNAs against MTHFD2. After 48 hours, relative cell viability was determined using the CCK8 assay. C. Bar graph showing quantification of PDX derived organoids representing the number of organoids per field in FIG. 1H. D. Nanoliposomal MTHFD2 siRNA delivery did not cause any loss in mice body weight during the course of experiment.

FIG. 5: Expression of MTHFD1L and MTHFD2L was assessed in various PCa cohorts. N: Normal, P: Primary, M: Metastatic.

FIG. 6: Graph providing data on identification of potent MTHFD2 siRNAs.

### DEFINITIONS

Unless defined otherwise, all technical and scientific terms used herein have the same meaning as is commonly understood by one of skill in the art to which the claimed subject matter.

As used herein, the singular forms “a,” “an” and “the” include plural referents unless the context clearly dictates otherwise.

As used herein, “subject” means a human or non-human animal selected for treatment or therapy.

As used herein, “in need thereof” means a subject identified as in need of a therapy or treatment.

As used herein, “administering” means providing a pharmaceutical agent or composition to a subject, and includes, but is not limited to, administering by a medical professional and self-administering. As used herein, “parenteral administration,” means administration through injection or infusion. Parenteral administration includes, but is not limited to, subcutaneous administration, intravenous administration, intraarticular or intramuscular administration.

As used herein, “subcutaneous administration” means administration just below the skin.

As used herein, “intravenous administration” means administration into a vein.

As used herein, “therapy” means a disease treatment method.

As used herein, “treatment” means the application of one or more specific procedures used for the cure or amelioration of a disease. In some embodiments, the specific procedure is the administration of one or more pharmaceutical agents.

As used herein, “amelioration” means a lessening of severity of at least one indicator of a condition or disease. In some embodiments, amelioration includes a delay or slowing in the progression of one or more indicators of a condition or disease. The severity of indicators may be determined by subjective or objective measures which are known to those skilled in the art.

As used herein, “prevention” refers to delaying or forestalling the onset or development or progression of a condition or disease for a period of time, including weeks, months, or years.

As used herein, “therapeutic agent” means a pharmaceutical agent used for the cure, amelioration or prevention of a disease.

As used herein, “dosage unit” means a form in which a pharmaceutical agent is provided. In some embodiments, a dosage unit is a vial containing lyophilized oligonucleotide. In some embodiments, a dosage unit is a vial containing reconstituted oligonucleotide.

As used herein, “therapeutically effective amount” refers to an amount of a pharmaceutical agent that provides a therapeutic benefit to an animal.

As used herein, “pharmaceutical composition” means a mixture of substances suitable for administering to a subject that includes a pharmaceutical agent. For example, a pharmaceutical composition may comprise a modified oligonucleotide and a sterile aqueous solution.

As used herein, “pharmaceutical agent” means a substance that provides a therapeutic effect when administered to a subject.

As used herein, “active pharmaceutical ingredient” means the substance in a pharmaceutical composition that provides a desired effect.

As used herein, “targeting” means the process of design and selection of nucleobase sequence that will hybridize to a target nucleic acid and induce a desired effect.

As used herein, “targeted to” means having a nucleobase sequence that will allow hybridization to a target nucleic acid to induce a desired effect. In some embodiments, a desired effect is reduction of a target nucleic acid.

As used herein, “modulation” means to a perturbation of function or activity. In some embodiments, modulation means an increase in gene expression. In some embodiments, modulation means a decrease in gene expression.

As used herein, “expression” means any functions and steps by which a gene's coded information is converted into structures present and operating in a cell.

As used herein, “nucleobase sequence” means the order of contiguous nucleobases, in a 5′ to 3′ orientation, independent of any sugar, linkage, and/or nucleobase modification.

As used herein, “contiguous nucleobases” means nucleobases immediately adjacent to each other in a nucleic acid.

As used herein, “percent identity” means the number of nucleobases in first nucleic acid that are identical to nucleobases at corresponding positions in a second nucleic acid, divided by the total number of nucleobases in the first nucleic acid. Percent identity between particular stretches of nucleotide sequences within nucleic acid molecules or amino acid sequences within polypeptides can be determined routinely using BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). Herein, if reference is made to percent sequence identity, the higher percentages of sequence identity are preferred over the lower ones.

As used herein, “hybridize” means the annealing of complementary nucleic acids that occurs through nucleobase complementarity.

As used herein, “mismatch” means a nucleobase of a first nucleic acid that is not capable of pairing with a nucleobase at a corresponding position of a second nucleic acid.

As used herein, “identical” means having the same nucleobase sequence.

As used herein, “oligomeric compound” means a compound comprising a polymer of linked monomeric subunits.

As used herein, “oligonucleotide” means a polymer of linked nucleosides, each of which can be modified or unmodified, independent from one another.

As used herein, “naturally occurring internucleoside linkage” means a 3′ to 5′ phosphodiester linkage between nucleosides.

As used herein, “natural sugar” means a sugar found in DNA (2′-H) or RNA (2′-OH).

As used herein, “natural nucleobase” means a nucleobase that is unmodified relative to its naturally occurring “internucleoside linkage” means a covalent linkage between adjacent nucleosides.

As used herein, “linked nucleosides” means nucleosides joined by a covalent linkage. As used herein, “nucleobase” means a heterocyclic moiety capable of non-covalently pairing with another nucleobase.

As used herein, “nucleoside” means a nucleobase linked to a sugar.

As used herein, “nucleotide” means a nucleoside having a phosphate group covalently linked to the sugar portion of a nucleoside.

As used herein, “modified oligonucleotide” means an oligonucleotide having one or more modifications relative to a naturally occurring terminus, sugar, nucleobase, and/or internucleoside linkage.

As used herein, “modified internucleoside linkage” means any change from a naturally occurring internucleoside linkage.

As used herein, “phosphorothioate internucleoside linkage” means a linkage between nucleosides where one of the non-bridging atoms is a sulfur atom.

As used herein, “modified sugar” means substitution and/or any change from a natural sugar.

As used herein, “modified nucleobase” means any substitution and/or change from a natural nucleobase.

As used herein, “5-methylcytosine” means a cytosine modified with a methyl group attached to the 5′ position.

As used herein, “2′-O-methyl sugar” or “2′-O-Me sugar” means a sugar having an O-methyl modification at the 2′ position.

As used herein, “2′-O-methoxyethyl sugar” or “2′-MOE sugar” means a sugar having a O-methoxyethyl modification at the 2′ position.

As used herein, “2′-O-fluoro” or “2′-F” means a sugar having a fluoro modification of the 2′ position.

As used herein, “bicyclic sugar moiety” means a sugar modified by the bridging of two non-geminal ring atoms.

As used herein, “2′-O-methoxyethyl nucleoside” means a 2′-modified nucleoside having a 2′-0-methoxyethyl sugar modification.

As used herein, “2′-fluoro nucleoside” means a 2′-modified nucleoside having a 2′-fluoro sugar modification.

As used herein, “2′-O-methyl” nucleoside means a 2′-modified nucleoside having a 2′-O-methyl sugar modification.

As used herein, “bicyclic nucleoside” means a 2′-modified nucleoside having a bicyclic sugar moiety. As used herein, “motif” means a pattern of modified and/or unmodified nucleobases, sugars, and/or internucleoside linkages in an oligonucleotide.

As used herein, a “fully modified oligonucleotide” means each nucleobase, each sugar, and/or each internucleoside linkage is modified.

As used herein, a “uniformly modified oligonucleotide” means each nucleobase, each sugar, and/or each internucleoside linkage has the same modification throughout the modified oligonucleotide.

As used herein, a “stabilizing modification” means any modification to a nucleoside that provides enhanced stability to a modified oligonucleotide relative to that provided by a conventional. For example, in some embodiments, a stabilizing modification is a stabilizing nucleoside modification. In some embodiments, a stabilizing modification is an internucleoside linkage modification.

As used herein, a “stabilizing nucleoside” means a nucleoside modified to provide enhanced nuclease stability to an oligonucleotide. In one embodiment, a stabilizing nucleoside is a 2′-modified nucleoside.

As used herein, a “stabilizing internucleoside linkage” means an internucleoside linkage that provides enhanced nuclease stability to an oligonucleotide relative to that provided by a phosphodiester internucleoside linkage. In one embodiment, a stabilizing internucleoside linkage is a phosphorothioate internucleoside linkage.

### DETAILED DESCRIPTION OF THE INVENTION

The present invention relates to the use of oligonucleotides to treat cancer, and in particular to treat prostate cancer.

Cell proliferation requires energy, the availability of building blocks for new cellular components, and the ability to maintain cellular redox homeostasis (Locasale J W, Cantley L C. Metabolic flux and the regulation of mammalian cell growth. Cell metabolism 2011; 14:443-51). For building block generation and redox homeostasis, amino acid metabolism involving serine and glycine, and the carbon units that they provide, are essential (Locasale J W. Serine, glycine and one-carbon units: cancer metabolism in full circle. Nat Rev Cancer 2013; 13:572-83). The 1C cycle mediates the folate-mediated transfer of 1C units from donor molecules, mainly serine, to acceptor molecules, such as purines, methionine and thymidylate; this is necessary for essential cellular processes including DNA synthesis, DNA repair, and the maintenance of cellular redox status.

Eukaryotic cells have complementary pathways for IC metabolism in the cytosol and mitochondria comprising distinct serine hydroxymethyltransferases (SHMTs) and methy lenetetrahydrofolate dehydrogenases (MTHFDs). While the cytoplasmic IC pathway (c1C) prevails in non-proliferating somatic tissues, the mitochondrial pathway (m1C) is predominantly active in proliferating cells, as well as in cancer cells (Meiser J, Vazquez. A. Give it or take it: the flux of one-carbon in cancer cells. FEBS J 2016; 283:3695-704). In fact, the central player of the m1C cycle, methylenetetrahydrofolate dehydrogenase 2 (MTHFD2), is overexpressed in many different tumor types (Nilsson R, Jain M, Madhusudhan N, Sheppard N G, Strittmatter L, Kampf C, et al. Metabolic enzyme expression highlights a key role for MTHFD2 and the mitochondrial folate pathway in cancer. Nat Commun 2014; 5:3128). MTHFD2 is also critical during embryonic development (Di Pietro E, Sirois J, Tremblay M L, Mackenzie R E. Mitochondrial NAD-dependent methylenetetrahydrofolate dehydrogenase-methenyltetrahydrofolate cyclohydrolase is essential for embryonic development. Molecular and cellular biology 2002; 22:4158-66), but is typically not expressed in normal adult tissues, except in highly proliferative cells, such as during T-cell lymphocyte activation (Nilsson et al., supra; Vazquez. A, Tedeschi P M, Bertino J R. Overexpression of the mitochondrial folate and glycine-serine pathway: a new determinant of methotrexate selectivity in tumors. Cancer Res 2013; 73:478-82).

The androgen receptor (AR) plays a key role in normal prostate growth, as well as in prostate carcinogenesis and progression. We previously found that AR signaling, a central driver of PCa, increased expression of activating transcription factor 4 (ATF4) (Sheng X, Arnoldussen Y J, Storm M, Tesikova M, Nenseth H Z, Zhao S, et al. Divergent androgen regulation of unfolded protein response pathways drives prostate cancer. EMBO Mol Med 2015; 7:788-801). We have recently found that ATF4 has essential pro-survival functions in PCa cells in vitro and in vivo through direct activation of a broad range of genes including key metabolic pathways (Pallmann N, Livgard M, Tesikova M, Zeynep Nenseth H, Akkus E, Sikkeland J, et al. Regulation of the unfolded protein response through ATF4 and FAM129A in prostate cancer. Oncogene 2019; 38:6301-18).

Experiments described herein identified MTHFD2 as a therapeutic target in cancer (e.g., prostate cancer). Accordingly, described herein are compositions and methods for targeting MTHFD2 in cancers. In particular, provided herein are nucleic acids (e.g., siRNAs) for decreasing expression of MTHFD2 and the use of such nucleic acids in the treatment of cancer (e.g., prostate cancer).

Accordingly, the present disclosure provides oligonucleotides, such as modified oligonucleotides, wherein the oligonucleotides, or a salt thereof, comprise a nucleobase sequence at least 80% identical to one of SEQ ID NOs: 1-63 and 126 to 140 or the complement thereof. Unless otherwise indicated, the sequences represented by the SEQ ID NOs are in 5′ to 3′ order.

In some preferred embodiments, the oligonucleotides, or a salt thereof, comprise a nucleobase sequence at least 90% identical to SEQ ID NO: 1 to 63 and 126 to 140 or the complement thereof. In some preferred embodiments, the oligonucleotides, or a salt thereof, comprise a nucleobase sequence at least 95% identical to SEQ ID NOs: 1 to 63 and 126 to 140 or the complement thereof. In some preferred embodiments, the oligonucleotides, or a salt thereof, binds to MTHFD2 (e.g., as described by SEQ ID NO:64) or an mRNA encoded by SEQ ID NO:64.

In still further preferred embodiments, the oligonucleotides consist of the linked nucleobase sequence corresponding to any one of SEQ ID NOs: 1 to 63 or 126 to 140 and/or the complement thereof. In some embodiments, the oligonucleotides consist essentially of the linked nucleobase sequence corresponding to any one of SEQ ID NOs: 1 to 63 or 126 to 140 operably linked to its complementary sequence so that it forms a double-stranded sequence.

In some embodiments, an oligonucleotide (such as an antisense oligonucleotide) consists of 15 to 30 linked nucleobases. In some embodiments, an oligonucleotide consists of 19 to 24 linked nucleobases. In some embodiments, an oligonucleotide consists of 21 to 24 linked nucleobases. In some embodiments, an oligonucleotide consists of 22 linked nucleobases. In some embodiments, the oligonucleotide consists of 15, 16, 17, 18, 19, 20, 21, 22, 23, 24, 25, 26, 27, 28, 29, 30, 31, 32, 33, 34, 35, 36, 37, 38, 39, or 40 linked nucleobases. In some embodiments, the oligonucleotide consists of 15, 16, 17, 18, 19, 20, 21, 22, 23, 24, 25, 26, 27, 28, 29, or 30 linked nucleobases. In some embodiments, the oligonucleotide consists of 15, 16, 17, 18, 19, 20, 21, 22, 23, 24, or 25 linked nucleobases. In some embodiments, the oligonucleotide comprises a nucleobase sequence comprising at least 16, at least 17, at least 18, at least 19, at least 20, at least 21, or at least 22, or at least 23 contiguous nucleobases of a nucleobase sequence of any one of SEQ ID NOs: 1 to 63 and 126 to 140 or the complement thereof. In some preferred embodiments, the oligonucleotides further comprise the complementary strand of the designated oligonucleotide (e.g., SEQ ID NOs: 1 to 63 and 126 to 140) so that a double-stranded structure can be formed.

In some embodiments, the nucleobase sequence of the oligonucleotide has no more than three mismatches compared to the nucleobase sequences of any one of SEQ ID NOs: 1 to 63 and 126 to 140 or the complement thereof. In some embodiments, the nucleobase sequence of the oligonucleotide has no more than two mismatches compared to the nucleobase sequences of any one of SEQ ID NOs: 1 to 63 and 126 to 140 or the complement thereof. In some embodiments, the nucleobase sequence of the oligonucleotide has no more than one mismatch compared to the nucleobase sequence of any one of SEQ ID NOs: 1 to 63 and 126 to 140 or the complement thereof.

In some embodiments, the nucleobase sequence of the oligonucleotide has no mismatches compared to the nucleobase sequence corresponding to SEQ ID NOs: 1 to 63 and 126 to 140 or the complement thereof. In each of these embodiments, the oligonucleotide can be a modified oligonucleotide. In some preferred embodiments, the oligonucleotides are RNAs (e.g., siRNAs).

In “RNA interference.” or “RNAi,” a “small interfering RNA” or “short interfering RNA” or “siRNA” or “short hairpin RNA” or “shRNA” molecule, or “miRNA,” an RNAi (e.g., single strand, duplex, or hairpin) of nucleotides is targeted to a nucleic acid sequence of interest, for example, MTHFD2.

The terms “iRNA”, “RNAi agent,” “iRNA agent,”, “RNA interference agent” as used interchangeably herein, refer to an agent that contains RNA as that term is used herein, and which mediates the targeted cleavage of an RNA transcript via an RNA-induced silencing complex (RISC) pathway. iRNA directs the sequence-specific degradation of mRNA through a process known as RNA interference (RNAi). The iRNA modulates, e.g., inhibits, the expression of a MTHFD2 gene in a cell, e.g., a cell within a subject, such as a mammalian subject.

In one embodiment, an RNAi agent of the invention includes a single stranded RNA that interacts with a target RNA sequence, e.g., a MTHFD2 target mRNA sequence, to direct the cleavage of the target RNA. Without wishing to be bound by theory it is believed that long double stranded RNA introduced into cells is broken down into siRNA by a Type III endonuclease known as Dicer (Sharp et al. (2001) Genes Dev. 15:485). Dicer, a ribonuclease-III-like enzyme, processes the dsRNA into 19-23 base pair short interfering RNAs with characteristic two base 3′ overhangs (Bernstein, et al., (2001) Nature 409:363). The siRNAs are then incorporated into an RNA-induced silencing complex (RISC) where one or more helicases unwind the siRNA duplex, enabling the complementary antisense strand to guide target recognition (Nykanen, et al., (2001) Cell 107:309). Upon binding to the appropriate target mRNA, one or more endonucleases within the RISC cleave the target to induce silencing (Elbashir, et al., (2001) Genes Dev. 15:188). Thus, in one aspect the invention relates to a single stranded RNA (siRNA) generated within a cell and which promotes the formation of a RISC complex to effect silencing of the target gene, i.e., a MTHFD2 gene. Accordingly, the term “siRNA” is also used herein to refer to an iRNA as described above.

In certain embodiments, the RNAi agent may be a single-stranded siRNA (ssRNAi) that is introduced into a cell or organism to inhibit a target mRNA. Single-stranded RNAi agents bind to the RISC endonuclease, Argonaute 2, which then cleaves the target mRNA. The single-stranded siRNAs are generally 15-30 nucleotides and are chemically modified. The design and testing of single-stranded siRNAs are described in U.S. Pat. No. 8,101,348 and in Lima et al., (2012) Cell 150:883-894, the entire contents of each of which are hereby incorporated herein by reference. Any of the antisense nucleotide sequences described herein may be used as a single-stranded siRNA as described herein or as chemically modified by the methods described in Lima et al., (2012) Cell 150:883-894.

In certain embodiments, an “iRNA” for use in the compositions, uses, and methods of the invention is a double stranded RNA and is referred to herein as a “double stranded RNA agent.” “double stranded RNA (dsRNA) molecule.” “dsRNA agent.” or “dsRNA”. The term “dsRNA”, refers to a complex of ribonucleic acid molecules, having a duplex structure comprising two anti-parallel and substantially complementary nucleic acid strands, referred to as having “sense” and “antisense” orientations with respect to a target RNA, i.e., a MTHFD2 gene. In some embodiments of the invention, a double stranded RNA (dsRNA) triggers the degradation of a target RNA, e.g., an mRNA, through a post-transcriptional gene-silencing mechanism referred to herein as RNA interference or RNAi.

An “RNA duplex” or dsRNA refers to the structure formed by the complementary pairing between two regions of an RNA molecule. The RNA used in RNAi is “targeted” to a gene in that the nucleotide sequence of the duplex portion of the RNAi is complementary to a nucleotide sequence of the targeted gene. In certain embodiments, the RNAi is targeted to the sequence encoding a marker described herein. In some embodiments, the length of the RNAi is less than 30 base pairs. In some embodiments, the RNA can be 32, 31, 30, 29, 28, 27, 26, 25, 24, 23, 22, 21, 20, 19, 18, 17, 16, 15, 14, 13, 12, 11 or 10 base pairs in length. In some embodiments, the length of the RNAi is 19 to 32 base pairs in length. In certain embodiment, the length of the RNAi is 19 or 21 base pairs in length.

In some embodiments, RNAi comprises a hairpin structure. In addition to the duplex portion, the hairpin structure may contain a loop portion positioned between the two sequences that form the duplex. The loop can vary in length. In some embodiments the loop is 5, 6, 7, 8, 9, 10, 11, 12, 13, 14, 15, 16, 17, 18, 19, 20, 21, 22, 23, 24, 25, 26 or 27 nucleotides in length. In certain embodiments, the loop is 18 nucleotides in length. The hairpin structure can also contain 3′ and/or 5′ overhang portions. In some embodiments, the overhang is a 3′ and/or a 5′ overhang 0, 1, 2, 3, 4 or 5 nucleotides in length.

“miRNA” or “miR” means a non-coding RNA between 18 and 25 nucleobases in length which hybridizes to and regulates the expression of a coding RNA. In certain embodiments, a miRNA is the product of cleavage of a pre-miRNA by the enzyme Dicer. Examples of miRNAs are found in the miRNA database known as miRBase.

As used herein, Dicer-substrate RNAs (DsiRNAs) are chemically synthesized asymmetric 25-mer/27-mer duplex RNAs that have increased potency in RNA interference compared to traditional RNAi. Traditional 21-mer RNAi molecules are designed to mimic Dicer products and therefore bypass interaction with the enzyme Dicer. Dicer has been recently shown to be a component of RISC and involved with entry of the RNAi into RISC. Dicer-substrate RNAi molecules are designed to be optimally processed by Dicer and show increased potency by engaging this natural processing pathway. Using this approach, sustained knockdown has been regularly achieved using sub-nanomolar concentrations. (U.S. Pat. No. 8,084,599; Kim et al., Nature Biotechnology 23:222 2005; Rose et al., Nucleic Acids Res., 33:4140 2005).

The transcriptional unit of a “shRNA” is comprised of sense and antisense sequences connected by a loop of unpaired nucleotides. shRNAs are exported from the nucleus by Exportin-5, and once in the cytoplasm, are processed by Dicer to generate functional RNAi molecules. “miRNAs” stem-loops are comprised of sense and antisense sequences connected by a loop of unpaired nucleotides typically expressed as part of larger primary transcripts (pri-miRNAs), which are excised by the Drosha-DGCR8 complex generating intermediates known as pre-miRNAs, which are subsequently exported from the nucleus by Exportin-5, and once in the cytoplasm, are processed by Dicer to generate functional miRNAs or siRNAs.

“Artificial miRNA” or an “artificial miRNA shuttle vector”, as used herein interchangeably, refers to a primary miRNA transcript that has had a region of the duplex stem loop (at least about 9-20 nucleotides) which is excised via Drosha and Dicer processing replaced with the siRNA sequences for the target gene while retaining the structural elements within the stem loop necessary for effective Drosha processing. The term “artificial” arises from the fact the flanking sequences (e.g., about 35 nucleotides upstream and about 40 nucleotides downstream) arise from restriction enzyme sites within the multiple cloning site of the RNAi. As used herein the term “miRNA” encompasses both the naturally occurring miRNA sequences as well as artificially generated miRNA shuttle vectors.

The RNAi can be encoded by a nucleic acid sequence, and the nucleic acid sequence can also include a promoter. The nucleic acid sequence can also include a polyadenylation signal. In some embodiments, the polyadenylation signal is a synthetic minimal polyadenylation signal or a sequence of six Ts.

In some embodiments, the siRNA is provided as an expression vector encoding a shRNA comprising a nucleic acid that expresses an RNA selected from SEQ ID NOs: 1-63 and 126 to 140 linked to a nucleic acid encoding the complement of an RNA selected from SEQ ID NOs: 1-63 and 126 to 140. Such shRNAs are cleaved to form an siRNA in vivo.

In some preferred embodiments, the oligonucleotides are provided as double-stranded molecules (e.g. with a complementary strand) which may optionally be conjugated to a moiety facilitating cellular uptake. In some embodiments, the RNAi agent is linked to a ligand. Suitable ligands include, but are not limited to, lipid moieties, protein moieties including targeting moieties such as antibodies, lectins, and carbohydrates (e.g., N-acetylgalactosamine). Suitable ligands and methods for conjugation of the ligand to a RNAi agent are described in U.S. Pat. No. 11,326,166, the entire contents of which are incorporated herein by reference. In some preferred embodiments, at least 90%, 95% or 100% of the bases in said double-stranded molecules are in the form of 2′-Fluoro or 2′-Methoxy bases. In some preferred embodiments, the terminal nucleotides in the double-stranded molecules comprise a phosphorothioate linkage. Accordingly, in one embodiment, there is provided a double-stranded oligonucleotide wherein one strand is represented by the any one of SEQ ID NOs: 1 to 63 and 126 to 140 or sequences at least 80%, 90% or 95% identical to any one of SEQ ID NOs: 1 to 63 and 126 to 140 and wherein all of the bases are either 2′-Fluoro or 2′-Methoxy bases, and wherein the complementary strand is optionally conjugated to a moiety facilitating cellular uptake in the desired tissue and wherein the other terminal nucleotides comprise at least one phosphorothioate linkage. One example of a moiety facilitating cellular uptake is the triantennary N-acetylgalactosamine represented by the formula:

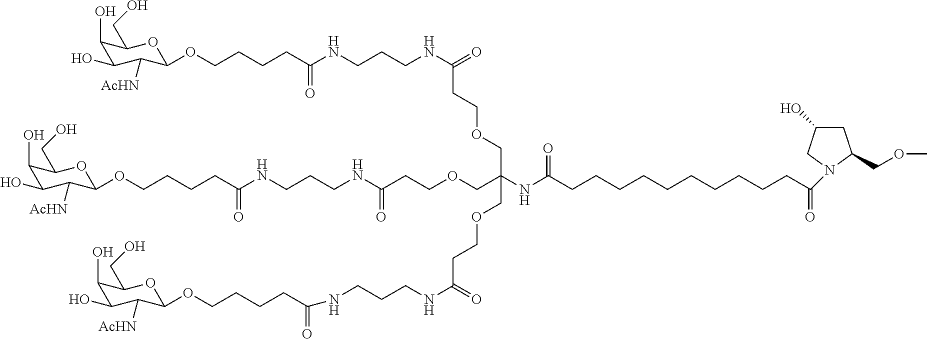 [SMILES: COC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

In some preferred embodiments, the oligonucleotides are provided as double-stranded molecules (e.g. with a complementary strand) which may optionally be conjugated to a moiety facilitating cellular uptake. In some preferred embodiments, at least 90%, 95% or 100% of the bases in said double-stranded molecules are in the form of 2′-Fluoro or 2′-Methoxy bases. In some preferred embodiments, the terminal nucleotides in the double-stranded molecules comprise a phosphorothioate linkage. Pharmaceutical compositions suitable for injection may preferably be isotonic, sterile solutions with a pH in the range of 6 to 8. The RNA oligonucleotides may be formulated into micelles or liposomes for facilitating cellular uptake. However, they may also be electroporated into target cells in situ or uptake into target cells may be facilitated via other, novel means. Such pharmaceutical compositions may provide an anti-inflammatory effect able to provide a prophylactic or therapeutic treatment of cancer.

It is contemplated that these RNA oligonucleotides have therapeutic utility in relation to cancer (e.g., prostate cancer).

The present invention is not limited to the use of any particular oligonucleotide formats. Suitable nucleic acids include, but are not limited to, deoxyribonucleic acid (DNA), ribonucleic acid (RNA), modified DNA or RNA, peptide nucleic acid (PNA), morpholino, locked nucleic acid (LNA), glycol nucleic acid (GNA), threose nucleic acid (TNA), DNA containing phosphorothioate residues (S-oligos) and derivatives thereof, or any combination thereof.

In some embodiments, one or more additional nucleobases may be added to either or both of the 3′ terminus and 5′ terminus of an oligonucleotide in comparison to the nucleobases sequences set forth in any of SEQ ID NOs: 126 to 140. In some embodiments, the one or more additional linked nucleobases are at the 3′ terminus. In some embodiments, the one or more additional linked nucleosides are at the 5′ terminus. In some embodiments, two additional linked nucleosides are linked to a terminus. In some embodiments, one additional nucleoside is linked to a terminus. In each of these embodiments, the oligonucleotide can be a modified oligonucleotide.

In some embodiments, the oligonucleotide comprises one or more modified internucleoside linkages, modified sugars, or modified nucleobases, or any combination thereof. Suitable modified linkages, sugars and nucleobases are described in U.S. Pat. No. 11,326,166, the entire contents of which are incorporated herein by reference. The nucleobase sequences set forth herein, including but not limited to those found in the Examples and in the sequence listing, are independent of any modification to the nucleic acid. As such, nucleic acids may comprise, independently, one or more modifications to one or more sugar moieties, to one or more internucleoside linkages, and/or to one or more nucleobases. A modified nucleobase, sugar, and/or internucleoside linkage may be selected over an unmodified form because of desirable properties such as, for example, enhanced cellular uptake, enhanced affinity for other oligonucleotides or nucleic acid targets and increased stability in the presence of nucleases.

In some embodiments, at least one internucleoside linkage is a modified internucleoside linkage. In some embodiments, each internucleoside linkage is a modified internucleoside linkage. In some embodiments, a modified internucleoside linkage comprises a phosphorus atom. In some embodiments, a modified oligonucleotide comprises at least one phosphorothioate internucleoside linkage. In some embodiments, each internucleoside linkage of a modified oligonucleotide is a phosphorothioate internucleoside linkage. In some embodiments, a modified internucleoside linkage does not comprise a phosphorus atom. In some such embodiments, an internucleoside linkage is formed by a short chain alkyl internucleoside linkage. In some such embodiments, an internucleoside linkage is formed by a cycloalkyl internucleoside linkages. In some such embodiments, an internucleoside linkage is formed by a mixed heteroatom and alkyl internucleoside linkage. In some such embodiments, an internucleoside linkage is formed by a mixed heteroatom and cycloalkyl internucleoside linkages. In some such embodiments, an internucleoside linkage is formed by one or more short chain heteroatomic internucleoside linkages. In some such embodiments, an internucleoside linkage is formed by one or more heterocyclic internucleoside linkages. In some such embodiments, an internucleoside linkage has an amide backbone. In some such embodiments, an internucleoside linkage has mixed N, O, S and CH₂ component parts.

In some embodiments, at least one nucleobase of the modified oligonucleotide comprises a modified sugar. In some embodiments, each of a plurality of nucleosides comprises a modified sugar. In some embodiments, each nucleoside of the modified oligonucleotide comprises a modified sugar. In each of these embodiments, the modified sugar may be a 2′-O-methoxyethyl sugar, a 2′-fluoro sugar, a 2′-O-methyl sugar, or a bicyclic sugar moiety. In some embodiments, each of a plurality of nucleosides comprises a 2′-O-methoxyethyl sugar and each of a plurality of nucleosides comprises a 2′-fluoro sugar.

In some embodiments, the sugar-modified nucleosides can further comprise a natural or modified heterocyclic base moiety and/or a natural or modified internucleoside linkage and may include further modifications independent from the sugar modification. In some embodiments, a sugar modified nucleoside is a 2′-modified nucleoside, wherein the sugar ring is modified at the 2′ carbon from natural ribose or 2′-deoxyribose.

In some embodiments, a 2′-modified nucleoside has a bicyclic sugar moiety. In some such embodiments, the bicyclic sugar moiety is a D sugar in the alpha configuration. In some such embodiments, the bicyclic sugar moiety is a D sugar in the beta configuration. In some such embodiments, the bicyclic sugar moiety is an L sugar in the alpha configuration. In some such embodiments, the bicyclic sugar moiety is an L sugar in the beta configuration. In some embodiments, the bicyclic sugar moiety comprises a bridge group between the 2′ and the 4′-carbon atoms. In some such embodiments, the bridge group comprises from 1 to 8 linked biradical groups. In some embodiments, the bicyclic sugar moiety comprises from 1 to 4 linked biradical groups. In some embodiments, the bicyclic sugar moiety comprises 2 or 3 linked biradical groups. In some embodiments, the bicyclic sugar moiety comprises 2 linked biradical groups. Biradical groups are well known in the art.

In some embodiments, the modified oligonucleotide comprises at least one modified nucleobase. In some embodiments, the modified nucleobase is selected from 5-hydroxymethyl cytosine, 7-deazaguanine and 7-deazaadenine. In some embodiments, the modified nucleobase is selected from 7-deaza-adenine, 7-deazaguanosine, 2-aminopyridine and 2-pyridone. In some embodiments, the modified nucleobase is selected from 5-substituted pyrimidines, 6-azapyrimidines and N-2, N-6 and O-6 substituted purines, including 2 aminopropyladenine, 5-propynyluracil and 5-propynylcytosine. In some embodiments, the modified nucleobase is a 5-methylcytosine. In some embodiments, at least one nucleoside comprises a cytosine, wherein the cytosine is a 5-methylcytosine. In some embodiments, each cytosine is a 5-methylcytosine.

In some embodiments, a 2′-modified nucleoside comprises a 2′-substituent group selected from halo, allyl, amino, azido, —SH, —CN, —OCN, —CF₃, —OCF₃, —O—, —S—, or —N(Rₘ)-alkyl; —O—, —S—, or —N(Rₘ)-alkenyl; —O—, —S— or —N(Rₘ)-alkynyl; —O-alkylenyl-O-alkyl, alkynyl, alkaryl, aralkyl, —O-alkaryl, —O-aralkyl, —O(CH₂)₂SCH₃, —O—(CH₂)₂—O—N(Rₘ)(Rₙ) or —O—CH₂—C(═O N(Rₘ)(Rₙ), where each Rₘ and Rₙ is, independently, H, an amino protecting group or substituted or unsubstituted Ci-ioalkyl. These 2′-substituent groups can be further substituted with one or more substituent groups independently selected from hydroxyl, amino, alkoxy, carboxy, benzyl, phenyl, nitro, thiol, thioalkoxy, halogen, alkyl, aryl, alkenyl and alkynyl.

In some embodiments, a 2′-modified nucleoside comprises a 2′-substituent group selected from F, NH₂, N₃, OCF₃, O—CH₃, O(CH₂); NH₂, CH₂—CH═CH₂, O—CH₂—CH═CH₂, OCH₂CH₂OCH₃, O(CH₂)₂SCH₃, O—(CH₂)₂—O—N(Rₘ)(Rₙ), —O(CH2)₂O(CH₂)₂N(CH₃)₂, and N-substituted acetamide (O—CH₂—C(═O)—N(Rm)(Rₙ) where each Rₘ and Rₙ is, independently, H, an amino protecting group or substituted or unsubstituted G_ioalkyl. In some embodiments, a 2′-modified nucleoside comprises a 2′-substituent group selected from F, OCF₃, O—CH₃, OCH₂CH₂OCH₃, 2′-O(CH₂)₂SCH₃, O—(CH₂)₂—O—N(CH₃)₂, —O(CH₂)₂O(CH₂)₂N(CH₃)₂, and O—CH₂—C(═O)—N(H)CH₃.

In some embodiments, a 2′-modified nucleoside comprises a 2′-substituent group selected from F, O—CH₃, and OCH₂CH₂OCH₃. In some embodiments, a sugar-modified nucleoside is a 4′-thio modified nucleoside. In some embodiments, a sugar-modified nucleoside is a 4′-thio-2′-modified nucleoside. A 4′-thio modified nucleoside has a B-D-ribonucleoside where the 4′-O replaced with 4′-S. A 4′-thio-2′-modified nucleoside is a 4′-thio modified nucleoside having the 2′-OH replaced with a 2′-substituent group. Suitable 2′-substituent groups include 2′-OCH₃, 2′-O—(CH₂)₂—OCH₃, and 2′-F.

In some embodiments, a modified nucleobase comprises a polycyclic heterocycle. In some embodiments, a modified nucleobase comprises a tricyclic heterocycle. In some embodiments, a modified nucleobase comprises a phenoxazine derivative. In some embodiments, the phenoxazine can be further modified to form a nucleobase known in the art as a G-clamp.

In some embodiments, the oligonucleotide compound comprises a modified oligonucleotide conjugated to one or more moieties which enhance the activity, cellular distribution or cellular uptake of the resulting antisense oligonucleotides. In some such embodiments, the moiety is a cholesterol moiety or a lipid moiety. Additional moieties for conjugation include carbohydrates, phospholipids, biotin, phenazine, folate, phenanthridine, anthraquinone, acridine, fluoresceins, rhodamines, coumarins, and dyes. In some embodiments, a conjugate group is attached directly to a modified oligonucleotide. In some embodiments, a conjugate group is attached to a modified oligonucleotide by a linking moiety selected from amino, hydroxyl, carboxylic acid, thiol, unsaturations (e.g., double or triple bonds), 8-amino-3,6-dioxaoctanoic acid (ADO), succinimidyl4-(N-maleimidomethyl)cyclohexane-1-carboxylate (SMCC), 6-aminohexanoic acid (AHEX or AHA), substituted G-ioalkyl, substituted or unsubstituted C2-ioalkenyl, and substituted or unsubstituted C2-ioalkynyl. In some such-embodiments, a substituent group is selected from hydroxyl, amino, alkoxy, carboxy, benzyl, phenyl, nitro, thiol, thioalkoxy, halogen, alkyl, aryl, alkenyl and alkynyl.

In some such embodiments, the oligonucleotide compound comprises a modified oligonucleotide having one or more stabilizing groups that are attached to one or both termini of a modified oligonucleotide to enhance properties such as, for example, nuclease stability. Included in stabilizing groups are cap structures. These terminal modifications protect a modified oligonucleotide from exonuclease degradation, and can help in delivery and/or localization within a cell. The cap can be present at the 5′-terminus (5′-cap), or at the 3′-terminus (3′-cap), or can be present on both termini. Cap structures include, for example, inverted deoxy abasic caps. Additional cap structures include, but are not limited to, a 4′,5′-methylene nucleotide, a 1-(beta-D-erythrofuranosyl) nucleotide, a 4′-thio nucleotide, a carbocyclic nucleotide, a 1,5-anhydrohexitol nucleotide, an L-nucleotide, an alpha-nucleotide, a modified base nucleotide, a phosphorodithioate linkage, a threopentofuranosyl nucleotide, an acyclic 3′,4′-seco nucleotide, an acyclic 3,4-dihydroxybutyl nucleotide, an acyclic 3,5-dihydroxypentyl nucleotide, a 3′-3′-inverted nucleotide moiety, a 3′-3′-inverted abasic moiety, a 3′-2′-inverted nucleotide moiety, a 3′-2′-inverted abasic moiety, a 1,4-butanediol phosphate, a 3′-phosphoramidate, a hexylphosphate, an aminohexyl phosphate, a 3′-phosphate, a 3′-phosphorothioate, a phosphorodithioate, a bridging methylphosphonate moiety, and a non-bridging methylphosphonate moiety 5′-amino-alkyl phosphate, a 1,3-diamino-2-propyl phosphate, 3-aminopropyl phosphate, a 6-aminohexyl phosphate, a 1,2-aminododecyl phosphate, a hydroxypropyl phosphate, a 5′-5′-inverted nucleotide moiety, a 5 ‘-5’-inverted abasic moiety, a 5′-phosphoramidate, a 5′-phosphorothioate, a 5′-amino, a bridging and/or non-bridging 5′-phosphoramidate, a phosphorothioate, and a 5′-mercapto moiety.

In particular, the present disclosure provides an oligonucleotide comprising 19 to 50 nucleobases, wherein the oligonucleotide optionally comprises at least one stabilizing modification, and wherein the oligonucleotide comprises a sequence represented by:

(SEQ ID NO: 126)
UAGACUUCAGCACUUCUCG,
(SEQ ID NO: 127)
UUUUUUGCAGCAAUAAUGG,
(SEQ ID NO: 128)
UUUCAUAAUUGUCUCACUG,
(SEQ ID NO: 129)
UUCUUCAACUGCUCUUUGG,
(SEQ ID NO: 130)
UCUCUCAUCAAUAUGCUCU,
(SEQ ID NO: 131)
AGCAAUAAUGGUAUUCUUC,
(SEQ ID NO: 132)
UUCAACAAUUCUUCCUCUG,
(SEQ ID NO: 133)
UUAAAUUCAACAAUUCUUC,
(SEQ ID NO: 134)
UAACAUGGAAUACUGAUCC,
(SEQ ID NO: 135)
UCAGAUUUGGAAUACCUGC,
(SEQ ID NO: 136)
UCCACAUCUCCAACCAACU,
(SEQ ID NO: 137)
UGAUAUACCCAGCUUUUUG,
(SEQ ID NO: 138)
UUAAGCUUUCAAUAAAUAC,
(SEQ ID NO: 139)
UAAUCACUAGGUUAAUGGC,
or
(SEQ ID NO: 140
UAUUGCAAUAGCACUUGGG.

Such oligonucleotides are anti-sense to the MTHFD2 and they may be present in siRNAs or shRNAs. The oligonucleotides can be synthesized chemically or via expression vectors in cellular systems.

In a particularly preferred embodiment, the present disclosure provides a double-stranded oligonucleotide, wherein the oligonucleotide optionally comprises at least one stabilizing modification and wherein one of the strands in the oligonucleotide sequence is represented by:

(SEQ ID NO: 35)
UAGACUUCAGCACUUCUCGCU,
(SEQ ID NO: 33)
UUUUUUGCAGCAAUAAUGGUA,
(SEQ ID NO: 5)
UUUCAUAAUUGUCUCACUGUU,
(SEQ ID NO: 20)
UUCUUCAACUGCUCUUUGGGA,
(SEQ ID NO: 13)
UCUCUCAUCAAUAUGCUCUGG,
(SEQ ID NO: 32)
AGCAAUAAUGGUAUUCUUCAU,
(SEQ ID NO: 8)
UUCAACAAUUCUUCCUCUGAA,
(SEQ ID NO: 9)
UUAAAUUCAACAAUUCUUCCU,
(SEQ ID NO: 17)
UAACAUGGAAUACUGAUCCAA,
(SEQ ID NO: 25)
UCAGAUUUGGAAUACCUGCAG,
(SEQ ID NO: 29)
UCCACAUCUCCAACCAACUUG,
(SEQ ID NO: 30)
UGAUAUACCCAGCUUUUUGUC,
(SEQ ID NO: 38)
UUAAGCUUUCAAUAAAUACAA,
(SEQ ID NO: 39)
UAAUCACUAGGUUAAUGGCUG,
or
(SEQ ID NO: 41)
UAUUGCAAUAGCACUUGGGAA.

In a particularly preferred embodiment, the present disclosure provides a siRNA comprising a pair of oligonucleotides represented by:

(SEQ ID NO: 99)
CGAGAAGUGCUGAAGUCUAAA
and
(SEQ ID NO: 35)
UAGACUUCAGCACUUCUCGCU,
(SEQ ID NO: 97)
CCAUUAUUGCUGCAAAAAAGG
and
(SEQ ID NO: 33)
UUUUUUGCAGCAAUAAUGGUA,
(SEQ ID NO: 69)
CAGUGAGACAAUUAUGAAACC
and
(SEQ ID NO: 5)
UUUCAUAAUUGUCUCACUGUU,
(SEQ ID NO: 84)
CCAAAGAGCAGUUGAAGAAAC
and
(SEQ ID NO: 20)
UUCUUCAACUGCUCUUUGGGA,
(SEQ ID NO: 77)
AGAGCAUAUUGAUGAGAGAAG
and
(SEQ ID NO: 13)
UCUCUCAUCAAUAUGCUCUGG,
(SEQ ID NO: 96)
GAAGAAUACCAUUAUUGCUGC
and
(SEQ ID NO: 32)
AGCAAUAAUGGUAUUCUUCAU,
(SEQ ID NO: 72)
CAGAGGAAGAAUUGUUGAAUU
and
(SEQ ID NO: 8)
UUCAACAAUUCUUCCUCUGAA,
(SEQ ID NO: 73)
GAAGAAUUGUUGAAUUUAAUC
and
(SEQ ID NO: 9)
UUAAAUUCAACAAUUCUUCCU,
(SEQ ID NO: 81)
GGAUCAGUAUUCCAUGUUACC
and
(SEQ ID NO: 17)
UAACAUGGAAUACUGAUCCAA,
(SEQ ID NO: 89)
GCAGGUAUUCCAAAUCUGAUC
and
(SEQ ID NO: 25)
UCAGAUUUGGAAUACCUGCAG,
(SEQ ID NO: 93)
AGUUGGUUGGAGAUGUGGAUU
and
(SEQ ID NO: 29)
UCCACAUCUCCAACCAACUUG,
(SEQ ID NO: 94)
CAAAAAGCUGGGUAUAUCACU
and
(SEQ ID NO: 30)
UGAUAUACCCAGCUUUUUGUC,
(SEQ ID NO: 102)
GUAUUUAUUGAAAGCUUAAAU
and
(SEQ ID NO: 38)
UUAAGCUUUCAAUAAAUACAA,
(SEQ ID NO: 103)
GCCAUUAACCUAGUGAUUAAU
and
(SEQ ID NO: 39)
UAAUCACUAGGUUAAUGGCUG,
or
(SEQ ID NO: 105)
CCCAAGUGCUAUUGCAAUAAC
and
(SEQ ID NO: 41)
UAUUGCAAUAGCACUUGGGAA.

Such pairs tend to form double-stranded oligonucleotides comprising 19 complementary base-pairs and a 2 nucleotide 3′overhang.

It will be appreciated that dsRNA agents of the instant invention include two RNA strands that are complementary and hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of an MTHFD2 gene. The other strand (the sense strand) includes a region that is complementary to the antisense strand, such that the two strands hybridize and form a duplex structure when combined under suitable conditions. As described elsewhere herein and as known in the art, the complementary sequences of a dsRNA can also be contained as self-complementary regions of a single nucleic acid molecule, as opposed to being on separate oligonucleotides.

Generally, the duplex structure is 15 to 30 base pairs in length, e.g., 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 base pairs in length. In certain embodiments, the duplex structure is 18 to 25 base pairs in length, e.g., 18-25, 18-24, 18-23, 18-22, 18-21, 18-20, 19-25, 19-24, 19-23, 19-22, 19-21, 19-20, 20-25, 20-24, 20-23, 20-22, 20-21, 21-25, 21-24, 21-23, 21-22, 22-25, 22-24, 22-23, 23-25, 23-24 or 24-25 base pairs in length, for example, 19-21 basepairs in length. Ranges and lengths intermediate to the above recited ranges and lengths are also contemplated to be part of the disclosure.

Similarly, the region of complementarity to the target sequence is 15 to 30 nucleotides in length, e.g., 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides in length, for example 19-23 nucleotides in length or 21-23 nucleotides in length. Ranges and lengths intermediate to the above recited ranges and lengths are also contemplated to be part of the disclosure.

In some embodiments, the duplex structure is 19 to 30 base pairs in length. Similarly, the region of complementarity to the target sequence is 19 to 30 nucleotides in length.

In some embodiments, the dsRNA is about 19 to about 23 nucleotides in length, or about 25 to about 30 nucleotides in length. In general, the dsRNA is long enough to serve as a substrate for the Dicer enzyme. For example, it is well-known in the art that dsRNAs longer than about 21-23 nucleotides in length may serve as substrates for Dicer. As the ordinarily skilled person will also recognize, the region of an RNA targeted for cleavage will most often be part of a larger RNA molecule, often an mRNA molecule. Where relevant, a “part” of an mRNA target is a contiguous sequence of an mRNA target of sufficient length to allow it to be a substrate for RNAi-directed cleavage (i.e., cleavage through a RISC pathway).

One of skill in the art will also recognize that the duplex region is a primary functional portion of a dsRNA, e.g., a duplex region of about 19 to about 30 base pairs, e.g., about 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 base pairs. Thus, in one embodiment, to the extent that it becomes processed to a functional duplex, of e.g., 15-30 base pairs, that targets a desired RNA for cleavage, an RNA molecule or complex of RNA molecules having a duplex region greater than 30 base pairs is a dsRNA. Thus, an ordinarily skilled artisan will recognize that in one embodiment, a miRNA is a dsRNA. In another embodiment, a dsRNA is not a naturally occurring miRNA. In another embodiment, an iRNA agent useful to target MTHFD2 gene expression is not generated in the target cell by cleavage of a larger dsRNA.

A dsRNA as described herein can further include one or more single-stranded nucleotide overhangs e.g., 1-4, 2-4, 1-3, 2-3, 1, 2, 3, or 4 nucleotides. dsRNAs having at least one nucleotide overhang can have superior inhibitory properties relative to their blunt-ended counterparts. A nucleotide overhang can comprise or consist of a nucleotide/nucleoside analog, including a deoxynucleotide/nucleoside. The overhang(s) can be on the sense strand, the antisense strand, or any combination thereof. Furthermore, the nucleotide(s) of an overhang can be present on the 5′-end, 3′-end, or both ends of an antisense or sense strand of a dsRNA.

A dsRNA can be synthesized by standard methods known in the art. Double stranded RNAi compounds of the invention may be prepared using a two-step procedure. First, the individual strands of the double stranded RNA molecule are prepared separately. Then, the component strands are annealed. The individual strands of the siRNA compound can be prepared using solution-phase or solid-phase organic synthesis or both. Organic synthesis offers the advantage that the oligonucleotide strands comprising unnatural or modified nucleotides can be easily prepared. Similarly, single-stranded oligonucleotides of the invention can be prepared using solution-phase or solid-phase organic synthesis or both.

Regardless of the method of synthesis, the siRNA preparation can be prepared in a solution (e.g., an aqueous or organic solution) that is appropriate for formulation. For example, the siRNA preparation can be precipitated and redissolved in pure double-distilled water, and lyophilized. The dried siRNA can then be resuspended in a solution appropriate for the intended formulation process.

In an aspect, a dsRNA of the invention includes at least two nucleotide sequences, a sense sequence and an antisense sequence. The antisense strand, with or without optional overhangs, is preferably selected from any of SEQ ID NOs: 1 to 63 and 126 to 140. Corresponding sense strands are provided in Example 1 below and in any event can readily be determined by one of skill in the art.

In this aspect, one of the two sequences is complementary to the other of the two sequences, with one of the sequences being substantially complementary to a sequence of an mRNA generated in the expression of a MTHFD2 gene. As such, in this aspect, a dsRNA will include two oligonucleotides, where one oligonucleotide is described as the antisense strand (e.g., SEQ ID NOs: 1 to 63 and 126 to 140) and the second oligonucleotide is described as the corresponding sense strand of the antisense strand.

In certain embodiments, the substantially complementary sequences of the dsRNA are contained on separate oligonucleotides. In other embodiments, the substantially complementary sequences of the dsRNA are contained on a single oligonucleotide.

It will be understood that, although the sequences in the Example may not be described as modified or conjugated sequences, the RNA of the iRNA of the invention e.g., a dsRNA of the invention, may comprise any one of the sequences set forth in the Examples that is un-modified, un-conjugated, or modified or conjugated differently than described therein. In other words, the invention encompasses dsRNA utilized in the Examples which are un-modified, un-conjugated, modified, or conjugated, as described herein.

The skilled person is well aware that dsRNAs having a duplex structure of about 20 to 23 base pairs, e.g., 21, base pairs have been hailed as particularly effective in inducing RNA interference (Elbashir et al., EMBO 2001, 20:6877-6888). However, others have found that shorter or longer RNA duplex structures can also be effective (Chu and Rana (2007) RNA 14:1714-1719; Kim et al. (2005) Nat Biotech 23:222-226). In the embodiments described above, by virtue of the nature of the oligonucleotide sequences provided in any of the Examples, dsRNAs described herein can include at least one strand of a length of minimally 21 nucleotides. It can be reasonably expected that shorter duplexes having any one of the exemplary sequences minus only a few nucleotides on one or both ends can be similarly effective as compared to the dsRNAs described above. Hence, dsRNAs having a sequence of at least 19, 20, or more contiguous nucleotides derived from any one of the exemplary sequences, and differing in their ability to inhibit the expression of a MTHFD2 gene by not more than about 5, 10, 15, 20, 25, or 30% inhibition from a dsRNA comprising the full sequence, are contemplated to be within the scope of the present invention.

In addition, the RNAs provided in the Examples identify sites in a MTHFD2 transcript that is susceptible to RISC-mediated cleavage. As such, the present invention further features iRNAs that target within one of these sites. As used herein, an iRNA is said to target within a particular site of an RNA transcript if the iRNA promotes cleavage of the transcript anywhere within that particular site. In some preferred embodiments, the iRNA targets a region that comprises nucleotides 1 to 1100 of SEQ ID NO:64. In some further preferred embodiments, such an iRNA will generally include at least about 19 contiguous nucleotides from any one of the sequences provided in the Examples coupled to additional nucleotide sequences taken from the region contiguous to the selected sequence in a MTHFD2 gene.

An RNAi agent as described herein can contain one or more mismatches to the target sequence. In one embodiment, an RNAi agent as described herein contains no more than 3 mismatches (i.e., 3, 2, 1, or 0) mismatches). In one embodiment, an RNAi agent as described herein contains no more than 2 mismatches. In one embodiment, an RNAi agent as described herein contains no more than 1 mismatch. In one embodiment, an RNAi agent as described herein contains 0 mismatches. In certain embodiments, if the antisense strand of the RNAi agent contains mismatches to the target sequence, the mismatch can optionally be restricted to be within the last 5 nucleotides from either the 5′- or 3′-end of the region of complementarity. For example, in such embodiments, for a 23 nucleotide RNAi agent, the strand which is complementary to a region of a MTHFD2 gene generally does not contain any mismatch within the central 13 nucleotides. The methods described herein or methods known in the art can be used to determine whether an RNAi agent containing a mismatch to a target sequence is effective in inhibiting the expression of a MTHFD2 gene. Consideration of the efficacy of RNAi agents with mismatches in inhibiting expression of a MTHFD2 gene is important, especially if the particular region of complementarity in a MTHFD2 gene is known to have polymorphic sequence variation within the population.

The present disclosure also provides pharmaceutical compositions comprising one or more of the oligonucleotides described herein. In some embodiments, the oligonucleotide consists of 15 to 40 linked nucleosides, or a salt thereof, wherein the modified oligonucleotide comprises a nucleobase sequence that is at least 80% identical to the nucleobase sequence of any one of SEQ ID NOs:1 to 63 and 126 to 140 as described in detail above and a pharmaceutically acceptable carrier or diluent. In each of these embodiments, the oligonucleotide can be a modified oligonucleotide.

In some embodiments, the compositions may additionally contain other adjunct components conventionally found in pharmaceutical compositions, at their art-established usage levels. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions, such as dyes, flavoring agents, preservatives, antioxidants, opacifiers, thickening agents and stabilizers. However, such materials, when added, should not unduly interfere with the biological activities of the components of the compositions. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the oligonucleotide(s) of the formulation.

In some embodiments, pharmaceutical compositions comprise one or more modified oligonucleotides and one or more excipients. In some such embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In some embodiments, a pharmaceutical composition is prepared using known techniques, including, but not limited to mixing, dissolving, granulating, dragee-making, levigating, emulsifying, encapsulating, entrapping or tab letting processes.

In some embodiments, a pharmaceutical composition is a liquid (e.g., a suspension, elixir and/or solution). In some such embodiments, a liquid pharmaceutical composition is prepared using ingredients known in the art, including, but not limited to, water, glycols, oils, alcohols, flavoring agents, preservatives, and coloring agents.

In some embodiments, a pharmaceutical composition is a solid (e.g., a powder, tablet, and/or capsule). In some such embodiments, a solid pharmaceutical composition comprising one or more oligonucleotides is prepared using ingredients known in the art, including, but not limited to, starches, sugars, diluents, granulating agents, lubricants, binders, and disintegrating agents.

In some embodiments, a pharmaceutical composition is formulated as a depot preparation. Some such depot preparations are typically longer acting than non-depot preparations. In some embodiments, such preparations are administered by implantation (for example subcutaneously or intramuscularly) or by intramuscular injection. In some embodiments, depot preparations are prepared using suitable polymeric or hydrophobic materials (for example an emulsion in an acceptable oil) or ion exchange resins, or as sparingly soluble derivatives, for example, as a sparingly soluble salt.

In some embodiments, a pharmaceutical composition comprises a delivery system. Examples of delivery systems include, but are not limited to, liposomes and emulsions. Delivery systems are useful for preparing pharmaceutical compositions including those comprising hydrophobic compounds. In some embodiments, some organic solvents such as dimethylsulfoxide are used. In some embodiments, presently available RNA packaging technology can be used to packing the RNA in lipid complexes and to deliver the RNA. The delivery system can also comprise nanoparticles or nano-complexes. The delivery system can also comprise bacterial mini-cells comprising the RNA.

In some embodiments, a pharmaceutical composition comprises one or more tissue-specific delivery molecules designed to deliver the one or more pharmaceutical agents to specific tissues or cell types. For example, in some embodiments, pharmaceutical compositions include liposomes coated with a tissue-specific antibody.

In some embodiments, a pharmaceutical composition comprises a cosolvent system. Some such co-solvent systems comprise, for example, benzyl alcohol, a nonpolar surfactant, a water-miscible organic polymer, and an aqueous phase. In some embodiments, such cosolvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycoBOO. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In some embodiments, a pharmaceutical composition comprises a sustained-release system. A non-limiting example of such a sustained-release system is a semi-permeable matrix of solid hydrophobic polymers. In some embodiments, sustained-release systems may, depending on their chemical nature, release pharmaceutical agents over a period of hours, days, weeks or months.

In some embodiments, a pharmaceutical composition is prepared for oral administration. In some such embodiments, a pharmaceutical composition is formulated by combining one or more compounds comprising any one or more of the oligonucleotides described herein with one or more pharmaceutically acceptable carriers. Some such carriers enable pharmaceutical compositions to be formulated as tablets, pills, dragees, capsules, liquids, gels, syrups, slurries, suspensions and the like, for oral ingestion by a subject. In some embodiments, pharmaceutical compositions for oral use are obtained by mixing oligonucleotide and one or more solid excipient. Suitable excipients include, but are not limited to, fillers, such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). In some embodiments, such a mixture is optionally ground and auxiliaries are optionally added. In some embodiments, pharmaceutical compositions are formed to obtain tablets or dragee cores. In some embodiments, disintegrating agents (e.g., cross-linked polyvinylpyrrolidone, agar, or alginic acid or a salt thereof, such as sodium alginate) are added.

In some embodiments, dragee cores are provided with coatings. In some such embodiments, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to tablets or dragee coatings.

In some embodiments, pharmaceutical compositions for oral administration are push-fit capsules made of gelatin. Some such push-fit capsules comprise one or more of the oligonucleotides described herein in admixture with one or more filler such as lactose, binders such as starches, and/or lubricants such as talc or magnesium stearate and, optionally, stabilizers. In some embodiments, pharmaceutical compositions for oral administration are soft, sealed capsules made of gelatin and a plasticizer, such as glycerol or sorbitol. In some soft capsules, one or more of the oligonucleotides described herein are be dissolved or suspended in suitable liquids, such as fatty oils, liquid paraffin, or liquid polyethylene glycols. In addition, stabilizers may be added.

In some embodiments, pharmaceutical compositions are prepared for buccal administration. Some such pharmaceutical compositions are tablets or lozenges formulated in conventional manner.

In some embodiments, a pharmaceutical composition is prepared for administration by injection (e.g., intravenous, intraperitoneal, subcutaneous, intramuscular, etc.). In some such embodiments, a pharmaceutical composition comprises a carrier and is formulated in aqueous solution, such as water or physiologically compatible buffers such as Hanks's solution, Ringer's solution, or physiological saline buffer. In some embodiments, other ingredients are included (e.g., ingredients that aid in solubility or serve as preservatives). In some embodiments, injectable suspensions are prepared using appropriate liquid carriers, suspending agents and the like. Some pharmaceutical compositions for injection are presented in unit dosage form, e.g., in ampoules or in multi-dose containers. Some pharmaceutical compositions for injection are suspensions, solutions or emulsions in oily or aqueous vehicles, and may contain formulatory agents such as suspending, stabilizing and/or dispersing agents. Some solvents suitable for use in pharmaceutical compositions for injection include, but are not limited to, lipophilic solvents and fatty oils, such as sesame oil, synthetic fatty acid esters, such as ethyl oleate or triglycerides, and liposomes. Aqueous injection suspensions may contain substances that increase the viscosity of the suspension, such as sodium carboxymethyl cellulose, sorbitol, or dextran. Optionally, such suspensions may also contain suitable stabilizers or agents that increase the solubility of the oligonucleotides described herein to allow for the preparation of highly concentrated solutions. In some embodiments, a pharmaceutical composition is prepared for transmucosal administration. In some such embodiments penetrants appropriate to the barrier to be permeated are used in the formulation. Such penetrants are generally known in the art. In some embodiments, a pharmaceutical composition is prepared for administration by inhalation. Some such pharmaceutical compositions for inhalation are prepared in the form of an aerosol spray in a pressurized pack or a nebulizer. Some such pharmaceutical compositions comprise a propellant, e.g., dichlorodifluoromethane, trichlorofluoromethane, dichlorotetrafluoroethane, carbon dioxide or other suitable gas. In some embodiments using a pressurized aerosol, the dosage unit may be determined with a valve that delivers a metered amount. In some embodiments, capsules and cartridges for use in an inhaler or insufflator may be formulated. Some such formulations comprise a powder mixture of one or more of the oligonucleotides described herein and a suitable powder base such as lactose or starch. In some embodiments, a pharmaceutical composition is prepared for rectal administration, such as a suppositories or retention enema. Some such pharmaceutical compositions comprise known ingredients, such as cocoa butter and/or other glycerides.

In some embodiments, a pharmaceutical composition is prepared for topical administration. Some such pharmaceutical compositions comprise bland moisturizing bases, such as ointments, creams, gels, liniments, lotions, and salves. Exemplary suitable ointment bases include, but are not limited to, petrolatum, petrolatum plus volatile silicones, and lanolin and water in oil emulsions. Exemplary suitable cream bases include, but are not limited to, cold cream and hydrophilic ointment.

In some embodiments, a pharmaceutical composition comprises a modified oligonucleotide in a therapeutically effective amount. In some embodiments, the therapeutically effective amount is sufficient to prevent, alleviate or ameliorate symptoms of a disease or to prolong the survival of the subject being treated. Determination of a therapeutically effective amount is well within the capability of those skilled in the art. In some embodiments, the pharmaceutical composition may further comprise at least one additional therapeutic agent.

The present invention provides methods of treating cancer (e.g., prostate cancer) comprising administering to a subject in need thereof one or more of the oligonucleotides described herein, and/or a pharmaceutical agent that induces the production of the one or more oligonucleotides.

In some embodiments, administration of an oligonucleotide comprises intra-articular administration, intravenous administration, subcutaneous administration, transdermal administration, intraperitoneal administration. In some particularly preferred embodiments, administration of an oligonucleotide comprises intra-articular administration.

In some embodiments, any one or more of the oligonucleotides described herein is administered at a dose selected from 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, and 800 mg. The oligonucleotide may be administered one per day, once per week, once per two weeks, once per three weeks, or once per four weeks.

In some preferred embodiments, the administration of an oligonucleotide of the present invention results in relief or amelioration of one or more symptoms of prostate cancer.

In some embodiments, such pharmaceutical compositions comprise any one or more of the oligonucleotides or modified oligonucleotides described herein in a dose selected from 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg 125 mg 130 mg 135 mg 140 mg, 145 mg, 150 mg, 155 mg, 160 mg, 165 mg, 170 mg, 175 mg 180 mg 185 mg 190 mg 195 mg, 200 mg, 205 mg, 210 mg, 215 mg, 220 mg, 225 mg, 230 mg 235 mg 240 mg 245 mg, 250 mg, 255 mg, 260 mg, 265 mg, 270 mg, 270 mg, 280 mg, 285 mg 290 mg 295 mg 300 mg 305 mg, 310 mg, 315 mg, 320 mg, 325 mg, 330 mg, 335 mg, 340 mg 345 mg, 350 mg 355 mg 360 mg, 365 mg, 370 mg, 375 mg, 380 mg, 385 mg, 390 mg, 395 mg 400 mg 405 mg 410 mg 415 mg, 420 mg, 425 mg, 430 mg, 435 mg, 440 mg, 445 mg, 450 mg 455 mg, 460 mg 465 mg 470 mg, 475 mg, 480 mg, 485 mg, 490 mg, 495 mg, 500 mg, 505 mg 510 mg 515 mg 520 mg 525 mg, 530 mg, 535 mg, 540 mg, 545 mg, 550 mg, 555 mg, 560 mg 565 mg 570 mg 575 mg 580 mg, 585 mg, 590 mg, 595 mg, 600 mg, 605 mg, 610 mg, 615 mg 620 mg 625 mg 630 mg 635 mg, 640 mg, 645 mg, 650 mg, 655 mg, 660 mg, 665 mg, 670 mg 675 mg 680 mg 685 mg 690 mg, 695 mg, 700 mg, 705 mg, 710 mg, 715 mg, 720 mg, 725 mg 730 mg 735 mg 740 mg 745 mg, 750 mg, 755 mg, 760 mg, 765 mg, 770 mg, 775 mg, 780 mg 785 mg 790 mg 795 mg and 800 mg. In some such embodiments, a pharmaceutical composition comprises a dose of modified oligonucleotide selected from 25 mg, 50 mg, 75 mg, 100 mg, 150 mg, 200 mg, 250 mg, 300 mg. 350 mg, 400 mg, 500 mg, 600 mg, 700 mg, and 800 mg.

In some embodiments, a pharmaceutical agent is sterile lyophilized oligonucleotide that is reconstituted with a suitable diluent, e.g., sterile water for injection or sterile saline for injection. The reconstituted product is administered as a subcutaneous injection or as an intravenous infusion after dilution into saline. The lyophilized drug product consists of any one or more of the oligonucleotides or modified oligonucleotides described herein which has been prepared in water for injection, or in saline for injection, adjusted to pH 7.0-9.0 with acid or base during preparation, and then lyophilized. The lyophilized modified oligonucleotide may be 25-800 mg of any one or more of the oligonucleotides or modified oligonucleotides described herein. It is understood that this encompasses 25, 50, 75, 100, 125, 150, 175, 200, 225, 250, 275, 300, 325, 350, 375, 425, 450,475, 500, 525, 550,575, 600, 625, 650, 675, 700, 725, 750, 775, and 800 mg of modified lyophilized oligonucleotide. The lyophilized drug product may be packaged in a 2 mL Type I, clear glass vial (ammonium sulfate-treated), stoppered with a bromobutyl rubber closure and sealed with an aluminum FLIP-OFFR overseal.

The present disclosure also provides any one or more of the oligonucleotide compounds described herein, or compositions comprising the same, or methods of preparing the same, or methods of using the same, or uses any one or more of the oligonucleotide compounds described herein, or compositions comprising the same, substantially as described with reference to the accompanying examples and/or figures.

The present disclosure also provides any one or more of the oligonucleotide compounds described herein, or compositions comprising the same, for use in the manufacture of a medicament for treating cancer (e.g., prostate cancer).

The present disclosure also provides uses of any one or more of the oligonucleotide compounds described herein, or compositions comprising the same, for treating cancer (e.g., prostate cancer).

In some embodiments, the oligonucleotides described herein are administered in combination with one or more additional anti-cancer agents. Examples of anti-cancer therapies include targeted cancer therapy, surgery, chemotherapy, radiation therapy, immunotherapy/biological therapy, and photodynamic therapy.

Chemotherapeutic agents may also be used for the treatment of cancer. Examples of chemotherapeutic agents include alkylating agents, anti-metabolites, plant alkaloids and terpenoids, vinca alkaloids, podophyllotoxin, taxanes, topoisomerase inhibitors, and cytotoxic antibiotics. Cisplatin, carboplatin, and oxaliplatin are examples of alkylating agents. Other alkylating agents include mechlorethamine, cyclophosphamide, chlorambucil, ifosfamide. Alkylating agents may impair cell function by forming covalent bonds with the amino, carboxyl, sulfhydryl, and phosphate groups in biologically important molecules. Alternatively, alkylating agents may chemically modify a cell's DNA.

Biological therapy (sometimes called immunotherapy, biotherapy, or biological response modifier (BRM) therapy) uses the body's immune system, either directly or indirectly, to fight cancer or to lessen the side effects that may be caused by some cancer treatments. Biological therapies include interferons, interleukins, colony-stimulating factors, monoclonal antibodies, vaccines, gene therapy, and nonspecific immunomodulating agents.

In some embodiments, the biological therapy is immune checkpoint therapy. Immune checkpoint inhibitors target CTLA-4, PD-1, or PD-L1. Examples include but are not limited to, ipilimumab, nivolumab, pembrolizumab, spartalizumab, and atezolizumab.

In order that the subject matter disclosed herein may be more efficiently understood, examples are provided below. It should be understood that these examples are for illustrative purposes only and are not to be construed as limiting the claimed subject matter in any manner.

### EXPERIMENTAL

### Example 1

### Materials and Methods

### Cell Culture

293T, RWPE1, LNCaP, DU145, and 22Rv1 cell lines were purchased from the American Type Culture Collection (Rockville, MD). The VCaP, C4-2B, and LNCaP-c-MYC cell lines were kind gifts from Dr. Frank Smit (Radboud University Nijmegen Medical Centre, The Netherlands), Dr. Lelund Chung (Cedars-Sinai Medical Center, CA), and Dr. Ian G. Mills (Oslo University Hospital, Norway), respectively. Cells were routinely maintained in a humidified 5% CO2 and 95% air incubator at 37° C. PCa cells were cultured in RPMI 1640, and 293T cells in DMEM, containing 10% fetal calf serum, 50 U/ml penicillin, 50 μg/ml streptomycin, and 4 mM L-glutamine (all purchased from BioWhittaker-Cambrex). Where indicated, cells were treated with 30 nM Thapsigargin (Tg) (Sigma-Aldrich) for 5 h. unless stated otherwise. All cell lines were used within 15 passages after reviving from the frozen stocks and routinely tested and were free of mycoplasma contamination.

### Cell Proliferation and Viability Assays

Briefly, cells were reverse transfected using Lipofectamine RNAiMAX transfection reagent (ThermoFisher) and plated into 96-well or 6-well plates. Cells in 6-well plates were cultured for the indicated times, trypsinized, stained with trypan blue, and counted using a hemocytometer. The data shown are representative of at least three independent experiments performed in triplicate. Cells plated into the 96-well plates were cultured for 48 hrs and cell viability was measured using the CCK-8 kit (Bimake, Munich, Germany).

### Colony Formation and Prostatosphere Assays

Cells were trypsinized, seeded at a density of 5,000 cells per well into 6-well plates, and cultured for 2-3 weeks. The cells were then fixed with methanol and stained with 0.4% crystal violet. Colonies were quantified by extracting crystal violet in 10% acetic acid and measurement of absorbance at 590 nm. Prostatosphere assays were performed as described previously (Sheng X, Nenseth H Z, Qu S, Kuzu O F, Frahnow T, Simon L, et al. IRE1alpha-XBPIs pathway promotes prostate cancer by activating c-MYC signaling. Nat Commun 2019; 10:323). The data shown are representative of at least two independent experiments performed in triplicate.

### Quantitative PCR

RNA extraction, reverse transcription and quantitative polymerase chain reaction (qPCR) were performed as described previously (Sheng X, Arnoldussen Y J, Storm M. Tesikova M, Nenseth H Z, Zhao S, et al. Divergent androgen regulation of unfolded protein response pathways drives prostate cancer. EMBO Mol Med 2015; 7:788-801). The values were normalized to the relative amount of the internal standard GAPDH, TBP, or ACTB. Results normalized to GAPDH are presented unless indicated otherwise. PCR primer sequences are available upon request. The data shown are representative of at least two independent experiments performed in triplicate.

### Western Analysis

Whole-cell extracts and Western analyses were performed by standard methods as described previously (Sheng, 2015; supra). The antibodies used were: ATF4 (11815, Cell Signaling), ATF4 (A5514, Bimake), ASNS (146811AP) (Proteintech); MTHFD2 (sc-390708), GAPDH (sc-47274), β-Actin (sc-47778) (Santa Cruz. Biotechnology). All antibodies were used at a dilution of 1:1,000, except for MTHFD2 (1:100), GAPDH (1:5,000) and β-Actin (1:2,000). The data shown are representative of at least two independent experiments.

### Statistical Analysis

Mean and standard deviation values were calculated using Microsoft Excel software. The potential effects were evaluated using Student's two-sided t-test unless indicated otherwise. Values of p<0.05 were considered as significant. Statistically significant differences are denoted by *, **, and *** indicating p<0.05, p<0.01 and p<0.001, respectively. Error bars indicate SEM.

### Results

### MTHFD2 is Critical for PCa Cell Growth In Vitro and In Vivo

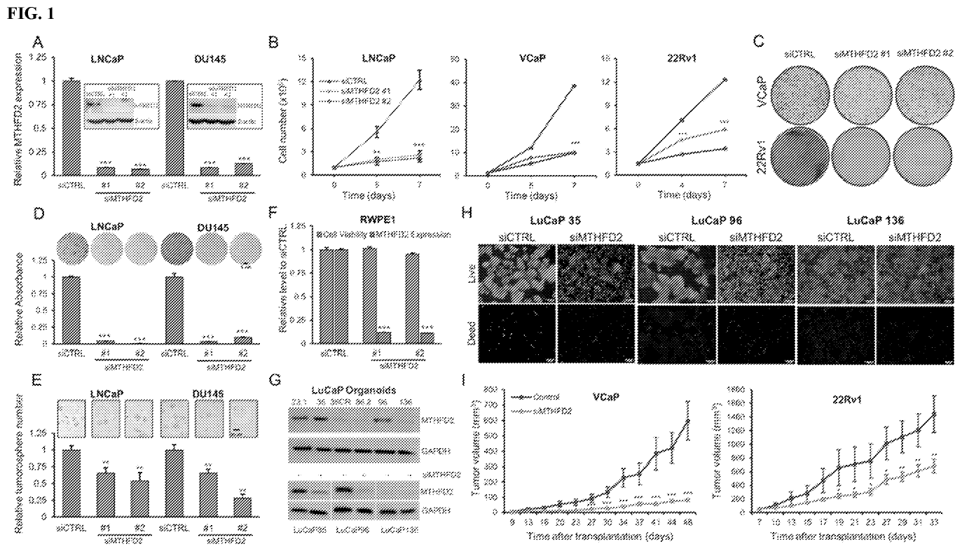

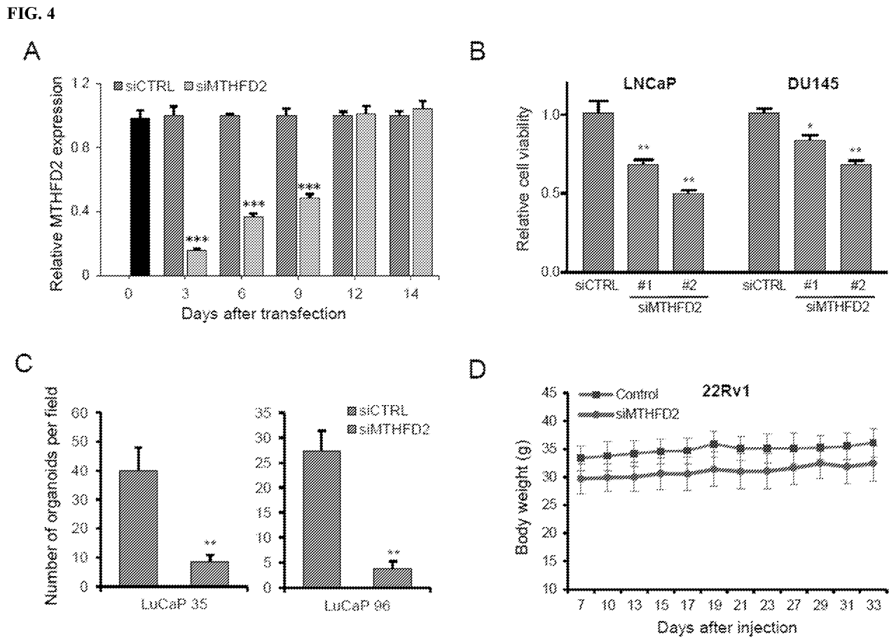

It was assessed whether MTHFD2 affects PCa cell growth. siRNA-mediated MTHFD2 silencing effectively reduced its mRNA and protein levels (FIG. 1A). siRNAs used were: siMTHFD2-1 TAGGATTATTCCTTGCTATTA (SEQ ID NO:62) and CAGGCCAGCTCAAGAAGCAAA (SEQ ID NO:63). MTHFD2 siMTHFD2-2 knockdown was maximal (˜80%) at 72 hours post-transfection and remained significantly downregulated for more than one week, but returned back to basal levels by day 12 (FIG. 4A). Short term MTHFD2 knockdown significantly reduced the viability (FIG. 4B) and long-term knockdown nearly abolished both viability and colony formation ability of LNCaP, DU145, VCaP, and 22Rv1 cells (FIGS. 1B-D). Furthermore, MTHFD2 knockdown significantly hindered LNCaP and DU145 prostatosphere growth (FIG. 1E). However, viability of a normal prostate epithelial cell line, RWPE1, was not affected by MTHFD2 knockdown (FIG. 1F).

To further evaluate the potential effects of MTHFD2 on PCa growth, organoids of LuCaP patient-derived xenograft (PDX) models were used (Nguyen H M, Vessella R L, Morrissey C, Brown L G, Coleman I M, Higano C S, et al. LuCaP Prostate Cancer Patient-Derived Xenografts Reflect the Molecular Heterogeneity of Advanced Disease and Serve as Models for Evaluating Cancer Therapeutics. Prostate 2017; 77:654-71). Three of the six analyzed LuCaP organoids expressed high levels of MTHFD2 expression (FIG. 1G). Three organoids, two with high expression and one with low expression, were analyzed further. These three PDX models, LuCaP 35, LuCaP 96, and LuCaP 136, express AR, lack PTEN expression, and were developed from lymph node metastasis, localized PCa, and adenocarcinoma cells from ascites, respectively (Nguyen H M, Vessella R L, Morrissey C, Brown L G, Coleman I M, Higano C S, et al. LuCaP Prostate Cancer Patient-Derived Xenografts Reflect the Molecular Heterogeneity of Advanced Disease and Serve as Models for Evaluating Cancer Therapeutics. Prostate 2017; 77:654-71). siRNA-mediated MTHFD2 knockdown effectively suppressed formation of LuCaP35 and LuCaP96 organoids that express high levels of MTHFD2 without inducing cell death (FIG. 1G-H). On the other hand, LuCaP 136 organoid that expresses very low levels of MTHFD2 was not affected by MTHFD2 knockdown.

To assess the therapeutic use of MTHFD2 inhibition in vivo, we performed xenograft experiments as previously described (Jin Y, Wang L, Qu S. Sheng X, Kristian A, Maelandsmo G M, et al. STAMP2 increases oxidative stress and is critical for prostate cancer. EMBO Mol Med 2015; 7:315-31; Jin Y, Qu S. Tesikova M, Wang L, Kristian A, Maelandsmo G M, et al. Molecular circuit involving KLK4 integrates androgen and mTOR signaling in prostate cancer. Proceedings of the National Academy of Sciences of the United States of America 2013; 110:E2572-81). VCaP or 22Rv1 cells were subcutaneously injected into male nude mice. Upon formation of palpable tumors, empty nanoliposomes or those that carry MTHFD2-specific siRNA were administered by intraperitoneal injection and tumor growth was monitored over time. Whereas tumors continued to grow rapidly in mice injected with the empty nanoliposomes, injection of nanoliposomes containing MTHFD2-specific siRNA dramatically inhibited tumor growth in both models (FIG. 4I). Nanoliposomal siMTHFD2 delivery was well-tolerated and did not result in any weight loss (FIG. 4D). Together with the findings from above, these data indicates that MTHFD2 is critical for PCa growth and serves as a therapeutic target.

### MTHFD2 Expression is Up-Regulated in Human PCa Specimens

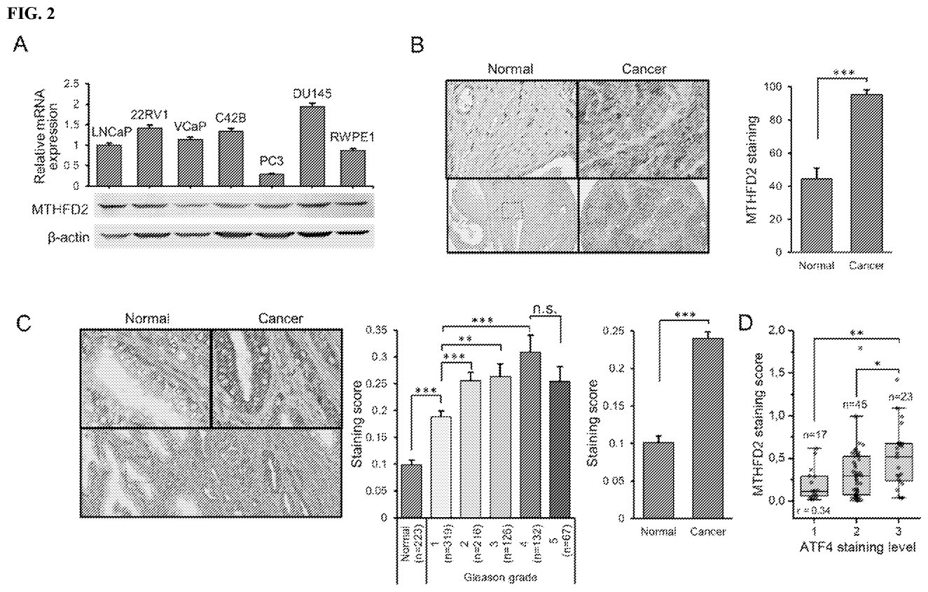

Both mRNA and protein expression of MTHFD2 were robust in the normal prostate cell line RWPE1 and in all of the PCa cell lines tested, with some variability in the level of expression (FIG. 2A). We next evaluated MTHFD2 expression in 24 human PCa specimens and their corresponding benign tissues from the same patients using immunohistochemistry. MTHFD2 expression was significantly increased in PCa compared to benign specimens (FIG. 2B). This observation was verified by an independent tissue microarray cohort consisting of 860 PCa and 223 benign prostate specimens (FIG. 2C). In this large cohort, MTHFD2 expression also correlated with the Gleason score indicating that it has prognostic value. Importantly, ATF4 and MTHFD2 protein expression was correlated in a sample subset of this TMA (FIG. 2D). These data show that MTHFD2 expression is significantly increased in PCa compared with normal tissue.

m1C Gene Expression Signature is Strongly Associated with PCa Prognosis

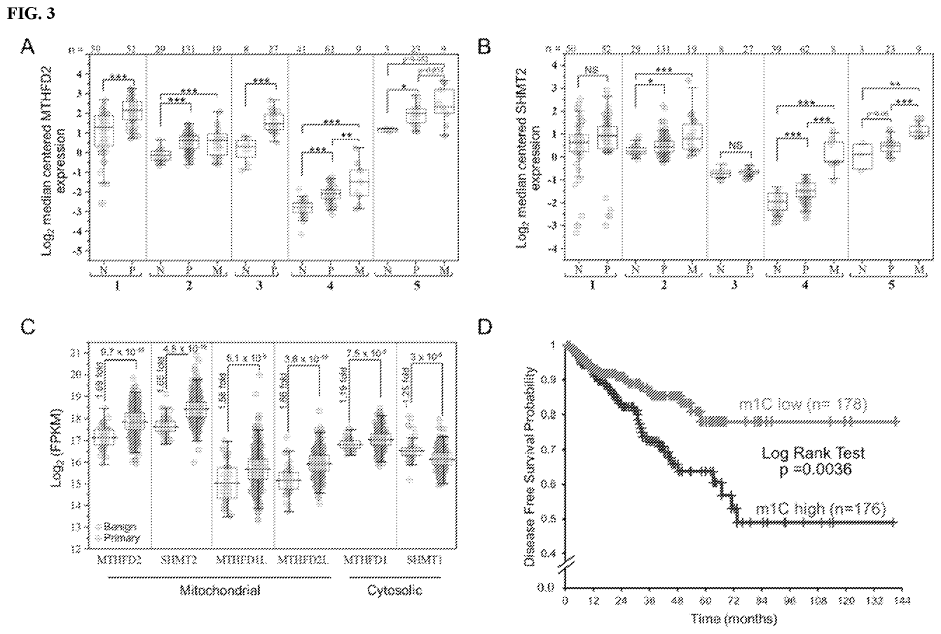

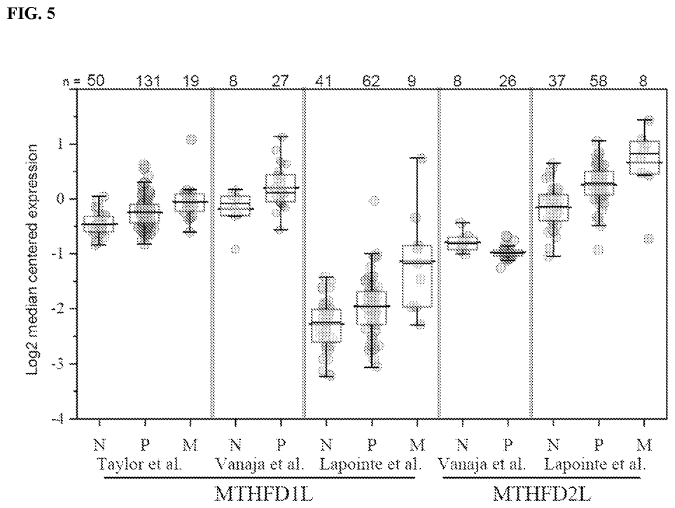

To assess whether ATF4-regulated m1C cycle gene expression could serve as a prognostic biomarker for PCa, we analyzed MTHFD2, SHMT2, MTHFD1L, and MTHFD2L expression in five independent PCa cohorts in the Oncomine database (Taylor B S, Schultz N, Hieronymus H, Gopalan A, Xiao Y, Carver B S, et al. Integrative genomic profiling of human prostate cancer. Cancer cell 2010; 18:11-22; Vanaja D K, Cheville J C, Iturria S J, Young C Y. Transcriptional silencing of zinc finger protein 185 identified by expression profiling is associated with prostate cancer progression. Cancer Res 2003; 63:3877-82; Lapointe J, Li C, Higgins J P, van de Rijn M, Bair E, Montgomery K, et al. Gene expression profiling identifies clinically relevant subtypes of prostate cancer. Proceedings of the National Academy of Sciences of the United States of America 2004; 101:811-6; LaTulippe E, Satagopan J, Smith A, Scher H, Scardino P, Reuter V, et al. Comprehensive gene expression analysis of prostate cancer reveals distinct transcriptional programs associated with metastatic disease. Cancer Res 2002; 62:4499-506; Singh D, Febbo P G, Ross K, Jackson D G, Manola J, Ladd C, et al. Gene expression correlates of clinical prostate cancer behavior. Cancer cell 2002; 1:203-9). MTHFD2 expression was consistently and significantly upregulated in primary and metastatic PCa compared to benign samples (FIG. 3A). SHMT2 was upregulated in three of the five cohorts (FIG. 3B). Only three cohorts had expression data for MTHFD1L and two for MTHFD2L. MTHFD1L was significantly upregulated in the primary and metastatic tumors while MTHFD2L was upregulated in one of the two cohorts (FIG. 5). In addition, in the Cancer Genome Atlas dataset, m1C metabolism gene expression, but not the cytosolic counterparts, were more prominently upregulated in primary tumor samples (FIG. 3C). These data led us to evaluate whether m1C metabolism gene expression may have prognostic value. Indeed, a gene expression signature consisting of the three m1C cycle enzymes (MTHFD2, MTHFD1L, and SHMT2) was significantly associated with recurrence-free survival in PCa patients (FIG. 3D). Taken together, these observations indicate that activation of ATF4-regulated m1C metabolism could serve as a prognostic biomarker in PCa.

### Example 2

Additional siRNAs that target MTHFD2 were developed. Two different approaches were used to generate the siRNAs:
1. Design based on the server-based implementation of the second-generation algorithm DSIR (biodev.extra.cea.fr/DSIR/DSIR.html) (Vert et al, 2006, BMC Bioinformatics) with a scoring threshold of 90/70 (original and corrected scores, respectively, default setting), and mismatch tolerance of 2.
2. Design by a combination of three first-generation algorithms (Reynold 2004, Amarzguioui 2004, Ui-Tei 2004), as implemented in Naito & Ui-Tei (2012, Frontiers in Genetics) server-based resource (sidirect2.rnai.jp/). SiRNAs that were indicated by all three algorithms, and did not overlap with those selected by the above method, were included.
   siRNAs were designed against coding region plus ˜3000 bp 3′-UTR of MTHFD2 transcript NM_006636.4 (www.ncbi.nlm.nih.gov/nuccore/NM_006636.4?report-genbank; SEQ ID NO:64).

All siRNAs were designed as standard 21-mers, with 2 nt ribonucleotide overhangs. siRNAs with GC content outside of 30-60% were excluded.

List of siRNAs:

All siRNAs receive a SEQ ID NO number name based on target position, from lowest to highest.

Target position refers to first base of passenger (sense) strand sequence.

| Pos. | Sequence/passenger/SEQ ID NO | AS Sequence/guide/SEQ ID NO |
|---|---|---|
| 88 | CGCGGCAGUUCGAAAUGAAGC (65) | UUCAUUUCGAACUGCCGCGAG (1) |
| 107 | GCUGUUGUCAUUUCUGGAAGG (66) | UUCCAGAAAUGACAACAGCUU (2) |
| 225 | GCGAGAAUCCUGCAAGUCACU (67) | UGACUUGCAGGAUUCUCGCCA (3) |
| 240 | GUCACUCCUAUGUCCUCAACA (68) | UUGAGGACAUAGGAGUGACUU (4) |
| 292 | CAGUGAGACAAUUAUGAAACC (69) | UUUCAUAAUUGUCUCACUGUU (5) |
| 313 | AGCUUCAAUUUCAGAGGAAGA (70) | UUCCUCUGAAAUUGAAGCUGG (6) |
| 318 | CAAUUUCAGAGGAAGAAUUGU (71) | AAUUCUUCCUCUGAAAUUGAA (7) |
| 324 | CAGAGGAAGAAUUGUUGAAUU (72) | UUCAACAAUUCUUCCUCUGAA (8) |
| 329 | GAAGAAUUGUUGAAUUUAAUC (73) | UUAAAUUCAACAAUUCUUCCU (9) |
| 336 | UGUUGAAUUUAAUCAAUAAAC (74) | UUAUUGAUUAAAUUCAACAAU (10) |
| 349 | CAAUAAACUGAAUAAUGAUGA (75) | AUCAUUAUUCAGUUUAUUGAU (11) |
| 358 | GAAUAAUGAUGAUAAUGUAGA (76) | UACAUUAUCAUCAUUAUUCAG (12) |
| 406 | AGAGCAUAUUGAUGAGAGAAG (77) | UCUCUCAUCAAUAUGCUCUGG (13) |
| 435 | AUGCUGUUUCUCCAGACAAGG (78) | UUGUCUGGAGAAACAGCAUUG (14) |
| 465 | GCUUUCAUGUAAUUAAUGUAG (79) | ACAUUAAUUACAUGAAAGCCA (15) |
| 489 | GAAUGUGUUUGGAUCAGUAUU (80) | UACUGAUCCAAACACAUUCGU (16) |
| 499 | GGAUCAGUAUUCCAUGUUACC (81) | UAACAUGGAAUACUGAUCCAA (17) |
| 676 | CACUGUUACAAUAUCUCAUCG (82) | AUGAGAUAUUGUAACAGUGGC (18) |
| 683 | ACAAUAUCUCAUCGAUAUACU (83) | UAUAUCGAUGAGAUAUUGUAA (19) |
| 705 | CCAAAGAGCAGUUGAAGAAAC (84) | UUCUUCAACUGCUCUUUGGGA (20) |
| 710 | GAGCAGUUGAAGAAACAUACA (85) | UAUGUUUCUUCAACUGCUCUU (21) |
| 718 | GAAGAAACAUACAAUUCUUGC (86) | AAGAAUUGUAUGUUUCUUCAA (22) |
| 725 | CAUACAAUUCUUGCAGAUAUU (87) | UAUCUGCAAGAAUUGUAUGUU (23) |
| 730 | AAUUCUUGCAGAUAUUGUAAU (88) | UACAAUAUCUGCAAGAAUUGU (24) |
| 758 | GCAGGUAUUCCAAAUCUGAUC (89) | UCAGAUUUGGAAUACCUGCAG (25) |
| 781 | AGCAGAUAUGAUCAAGGAAGG (90) | UUCCUUGAUCAUAUCUGCUGU (26) |
| 811 | CAUUGAUGUGGGAAUAAAUAG (91) | AUUUAUUCCCACAUCAAUGAC (27) |
| 830 | AGAGUUCACGAUCCUGUAACU (92) | UUACAGGAUCGUGAACUCUAU (28) |
| 861 | AGUUGGUUGGAGAUGUGGAUU (93) | UCCACAUCUCCAACCAACUUG (29) |
| 896 | CAAAAAGCUGGGUAUAUCACU (94) | UGAUAUACCCAGCUUUUUGUC (30) |
| 950 | GCAAUGCUAAUGAAGAAUACC (95) | UAUUCUUCAUUAGCAUUGCCA (31) |
| 961 | GAAGAAUACCAUUAUUGCUGC (96) | AGCAAUAAUGGUAUUCUUCAU (32) |
| 969 | CCAUUAUUGCUGCAAAAAAGG (97) | UUUUUUGCAGCAAUAAUGGUA (33) |
| 994 | GAGGCUUGAAGAGCGAGAAGU (98) | UUCUCGCUCUUCAAGCCUCAG (34) |
| 1007 | CGAGAAGUGCUGAAGUCUAAA (99) | UAGACUUCAGCACUUCUCGCU (35) |
| 1057 | GUGUCUUCUGUGUCACAAACA (100) | UUUGUGACACAGAAGACACAG (36) |
| 1121 | GCAAUAUUUUUAAUUUAUUCU (101) | AAUAAAUUAAAAAUAUUGCAU (37) |
| 1168 | GUAUUUAUUGAAAGCUUAAAU (102) | UUAAGCUUUCAAUAAAUACAA (38) |
| 1276 | GCCAUUAACCUAGUGAUUAAU (103) | UAAUCACUAGGUUAAUGGCUG (39) |
| 1323 | GGAUGCUUCACUUUGUCAAGC (104) | UUGACAAAGUGAAGCAUCCAU (40) |
| 1382 | CCCAAGUGCUAUUGCAAUAAC (105) | UAUUGCAAUAGCACUUGGGAA (41) |
| 2035 | AGUUUGAGUUACUAUUGAAUU (106) | UUCAAUAGUAACUCAAACUCU (42) |
| 2040 | GAGUUACUAUUGAAUUUAAUC (107) | UUAAAUUCAAUAGUAACUCAA (43) |
| 2149 | GACUCCUAGUUGUGACCUAAC (108) | UAGGUCACAACUAGGAGUCAU (44) |
| 2190 | CAAUGUAGCUAUUCCUAUAUU (109) | UAUAGGAAUAGCUACAUUGAU (45) |
| 2741 | GGCCUGUCCUACAGUCUUAUA (110) | UAAGACUGUAGGACAGGCCAG (46) |
| 2879 | GCUUGAGGCAGAAAGAGAAGA (111) | UUCUCUUUCUGCCUCAAGCUG (47) |
| 3047 | GCUUACCUAGAUUAUUAAAUG (112) | UUUAAUAAUCUAGGUAAGCAG (48) |
| 3075 | AGAUGACCAGACAGGAUUAAA (113) | UAAUCCUGUCUGGUCAUCUUG (49) |
| 3151 | GUAUUAUGUCAACCUGUUAAA (114) | UAACAGGUUGACAUAAUACUU (50) |
| 3205 | AAAUAUAAAUGACAUGUAAGG (115) | UUACAUGUCAUUUAUAUUUUG (51) |
| 3235 | AGUGAUCAAUUUACAACAAAG (116) | UUGUUGUAAAUUGAUCACUGU (52) |
| 3275 | AGCUUGUUUGAUUAAUCAAGC (117) | UUGAUUAAUCAAACAAGCUGG (53) |
| 3410 | GGAUUGGUGUGGAUGAUAAAU (118) | UUAUCAUCCACACCAAUCCUG (54) |
| 3741 | GACAAGAAUUUAUACUUAAGC (119) | UUAAGUAUAAAUUCUUGUCAG (55) |
| 3860 | GAGAUUAAAUGUAAUCAAACA (120) | UUUGAUUACAUUUAAUCUCAG (56) |
| 4047 | GGUUCAAGUGAUUUACUCAAG (121) | UGAGUAAAUCACUUGAACCUU (57) |
| 4123 | GGGUCAUAGUAGGCAUUAAAU (122) | UUAAUGCCUACUAUGACCCUG (58) |
| 4147 | CAUGGUUGGUGUUCAUUAAAU (123) | UUAAUGAACACCAACCAUGUU (59) |
| 4279 | GGUUGAUUAAUAUUGCAAAGU (124) | UUUGCAAUAUUAAUCAACCUA (60) |
| 4333 | CCUGUAUUGUUCACAUGAAUG (125) | UUCAUGUGAACAAUACAGGUU (61) |

>MTHFD2 cDNA_sequence (NM_006636.4, 1-4403)
(SEQ ID NO: 64)
CTCCCGGCGCAGTCACCGGCGCGGTCTATGGCTGCGACTTCTCTAATGT
CTGCTTTGGCTGCCCGGCTGCTGCAGCCCGCGCACAGCTGCTCCCTTCG
CCTTCGCCCTTTCCACCTCGCGGCAGTTCGAAATGAAGCTGTTGTCATT
TCTGGAAGGAAACTGGCCCAGCAGATCAAGCAGGAAGTGCGGCAGGAGG
TAGAAGAGTGGGTGGCCTCAGGCAACAAACGGCCACACCTGAGTGTGAT
CCTGGTTGGCGAGAATCCTGCAAGTCACTCCTATGTCCTCAACAAAACC
AGGGCAGCTGCAGTTGTGGGAATCAACAGTGAGACAATTATGAAACCAG
CTTCAATTTCAGAGGAAGAATTGTTGAATTTAATCAATAAACTGAATAA
TGATGATAATGTAGATGGCCTCCTTGTTCAGTTGCCTCTTCCAGAGCAT
ATTGATGAGAGAAGGATCTGCAATGCTGTTTCTCCAGACAAGGATGTTG
ATGGCTTTCATGTAATTAATGTAGGACGAATGTGTTTGGATCAGTATTC
CATGTTACCGGCTACTCCATGGGGTGTGTGGGAAATAATCAAGCGAACT
GGCATTCCAACCCTAGGGAAGAATGTGGTTGTGGCTGGAAGGTCAAAAA
ACGTTGGAATGCCCATTGCAATGTTACTGCACACAGATGGGGCGCATGA
ACGTCCCGGAGGTGATGCCACTGTTACAATATCTCATCGATATACTCCC
AAAGAGCAGTTGAAGAAACATACAATTCTTGCAGATATTGTAATATCTG
CTGCAGGTATTCCAAATCTGATCACAGCAGATATGATCAAGGAAGGAGC
AGCAGTCATTGATGTGGGAATAAATAGAGTTCACGATCCTGTAACTGCC
AAACCCAAGTTGGTTGGAGATGTGGATTTTGAAGGAGTCAGACAAAAAG
CTGGGTATATCACTCCAGTTCCTGGAGGTGTTGGCCCCATGACAGTGGC
AATGCTAATGAAGAATACCATTATTGCTGCAAAAAAGGTGCTGAGGCTT
GAAGAGCGAGAAGTGCTGAAGTCTAAAGAGCTTGGGGTAGCCACTAATT
AACTACTGTGTCTTCTGTGTCACAAACAGCACTCCAGGCCAGCTCAAGA
AGCAAAGCAGGCCAATAGAAATGCAATATTTTTAATTTATTCTACTGAA
ATGGTTTAAAATGATGCCTTGTATTTATTGAAAGCTTAAATGGGTGGGT
GTTTCTGCACATACCTCTGCAGTACCTCACCAGGGAGCATTCCAGTATC
ATGCAGGGTCCTGTGATCTAGCCAGGAGCAGCCATTAACCTAGTGATTA
ATATGGGAGACATTACCATATGGAGGATGGATGCTTCACTTTGTCAAGC
ACCTCAGTTACACATTCGCCTTTTCTAGGATTGCATTTCCCAAGTGCTA
TTGCAATAACAGTTGATACTCATTTTAGGTACCAAACCTTTTGAGTTCA
ACTGATCAAACCAAAGGAAAAGTGTTGCTAGAGAAAATTAGGGAAAAGG
TGAAAAAGAAAAAATGGTAGTAATTGAGCAGAAAAAAATTAATTTATAT
ATGTATTGATTGGCAACCAGATTTATCTAAGTAGAACTGAATTGGCTAG
GAAAAAAGAAAAACTGCATGTTAATCATTTTCCTAAGCTGTCCTTTTGA
GGCTTAGTCAGTTTATTGGGAAAATGTTTAGGATTATTCCTTGCTATTA
GTACTCATTTTATGTATGTTACCCTTCAGTAAGTTCTCCCCATTTTAGT
TTTCTAGGACTGAAAGGATTCTTTTCTACATTATACATGTGTGTTGTCA
TATTTGGCTTTTGCTATATACTTTAACTTCATTGTTAAATTTTTGTATT
GTATAGTTTCTTTGGTGTATCTTAAAACCTATTTTTGAAAAACAAACTT
GGCTTGATAATCATTTGGGCAGCTTGGGTAAGTACGCAACTTACTTTTC
CACCAAAGAACTGTCAGCAGCTGCCTGCTTTTCTGTGATGTATGTATCC
TGTTGACTTTTCCAGAAATTTTTTAAGAGTTTGAGTTACTATTGAATTT
AATCAGACTTTCTGATTAAAGGGTTTTCTTTCTTTTTTAATAAAACACA
TCTGTCTGGTATGGTATGAATTTCTGAAATTCTGTCTTCCTATGACTCC
TAGTTGTGACCTAACCAGTTTTTTTCTCATTTAATCAATGTAGCTATTC
CTATATTCAGTAACACTTACTTCTATAGCCTTAAATAGATAATTTTTTT
TCTTCTATTTTTTTTTTTTTTTTTGTAGAGATGGGGTCTTGCTATGTTG
CCTGGGCTGGTATTGAACTCCTGGCCTCAAGCAATCAATCCTCTTGCCT
CAGCTTCCCAGAGGGCTGGGATTACAGGCCTGGGCCACCCACACCCAAA
CAGAGGTTTAAAGAGCTTTGGAGAAACCAGCCTCGTCCTCCAGTCTCTT
TGTTTGTTTGTTTTGAGACTGAGTCTTGCTCTGTTGCCCAGGCTGGAGT
GCAGTGGCATGATCTCGGGTCACTGCAACCTCTGCCTCTCGGGTTCAAA
CAATTCTTGTGCCTCAGCCTCCTGAGTAGCTGGGATTACAGGTGCATGC
CACCATGCCTGGCTAATTTTTGTGTTTTTAGTAGAGATGGGGTTTCATC
ACATTGGCCAGGCTGGTCTCAAACTCCTGACCTCAAGTGATCCACCTGC
CTCGGCCTCCCAAAATGCTAGGATTACAGGCGTGAGCCACTGCACCTGG
CCTGTCCTACAGTCTTATATCACAACAGGTTGTGCCAAGCTAGGAGCTT
AATCTGTAAAATACTTGAGGCAGGGATAGGTTTGTCTGTTTTGGCTTTT
GGCTTTCATGCGAGCAGAAGTAGCTCCTGCTCTAGGCAGCTTGAGGCAG
AAAGAGAAGAAACAATTGAGTTGACCTACCTCCGTCCTCGTTCTTCTGC
CAGCTGGCGTTATTCTTTTGATAGTTCTCTGGAGTTTAAAGAAGTTTAG
ATAGTTTGCTGGAGATGTTTTTGTAAGTGACGTATAGCTTATGTCTCTA
ATCCATGCCTGCTTACCTAGATTATTAAATGTGGAGCAAGATGACCAGA
CAGGATTAAATTTTATTGAATTACAGTTACAAGTGGAAAAAATGACTGA
CTTACTGCATGGAGAAGTATTATGTCAACCTGTTAAAAGCTATGGAAAT
TAAAATGTTCATGTATATACAAAATATAAATGACATGTAAGGTAAAATT
ACAGTGATCAATTTACAACAAAGAAATTAACAGTAGTTTTCCAGCTTGT
TTGATTAATCAAGCAGGTTTACTCTTGAATCCATAGCATTTCTACAATT
TAATTTGGACAAAGTTCTATTCCAGATCAGCCAGGTTTTAAAACTGAAA
TGTGTTAAGGGTGGATTGTTAGCTTCCCCAGGATTGGTGTGGATGATAA
ATACTTTTTTTAAAAATTAATTATTATTGTTTATAAGACATGGATTCCA
GCTGTCTCCCTGGCTGGAGTGCAGTGGCATGATTATAGCTCACTGCAGC
CTTGAACTCCTGGACTTAAACGATCCTCCTGCATCAGCCTCTGAAATGG
CTGGGACTACAGGTGTGTGCCAGCATGCCTAGCTAATTTTTAAACATTT
GTTTAGAGATGGGTCTCACTATGTTGCCCAGGCTGGTCTTGACCTCCTG
ACCTCTGCTGATTCCCCCCACCTCTGCCTCCCAAGTAGCGGGATTACAG
GTGTGAGCCACTGTGTCTGACAAGAATTTATACTTAAGCATAGGAGATG
GTTCTGGAAATTCTAAGAAATTCTGCTCTCAGTAAGAGTAGAGGTTTGG
AGCTTTACCTCTTGGCAGTATCCCTTGGAAGGGAGCTCTGAGATTAAAT
GTAATCAAACACTTATTGAGAGCTTTCTATGGACCAGGCACTACACTAG
ACGCTGGGAATTACAGAGGCAATTACCTGTCCTATACTCAAGGTGTTAA
TGATCTAGTGTCATTTTTCTTTAAAAAAGAAAGCACCTGCATTTGTCAT
CTTGAGCAAATATAGTAACTCTAACCTCAAGGTTCAAGTGATTTACTCA
AGGTTTAAATCATAGTTGTTTTGGAGAAAGTAAGTGTCAGATGCACTGT
GGCTGTCAGGGTCATAGTAGGCATTAAATCAACATGGTTGGTGTTCATT
AAATGTGGATTGAAGATAAAATGACTGATCTTAAGTTTCATGAGGAATA
GGTTTGCTTCATTGAAACTGGTTTTTATCTTCCCAAATAGTTTTCAATC
ATTCCTTTAAAGTACTAGGTTGATTAATATTGCAAAGTTGTAATGTAAA
ATGTGACCTTACCTCTGTCTAACCTGTATTGTTCACATGAATGGGAGTT
TGACTTTGGGAATAAACATTGAAAATTTGCAGGGAGGACCCA

### Example 3

Using previously established siRNA design algorithms (DSIR and siDIRECT2), 60 siRNAs were designed as standard 21-mers, with 2 nt ribonucleotide overhangs to target full-length MTHFD2 mRNA (NM_006636.4). All siRNAs had a GC content between 30 to 60%. After an initial screen in two prostate cancer cell lines (LNCaP and C4-2B), efficiencies of the 41 most potent siRNAs were further evaluated in six different cancer cell lines (HCT116, A549, NCI-H460, C4-2B, 22Rv1, and MDA-MB-468) representing four distinct cancer types. Briefly, cells plated into 24 well plates were reverse transfected with 1 nM control or MTHFD2 siRNAs using RNAiMAX reagent (Thermo Fisher) according to the manufacturer's protocols. 48 hrs after transfections cells were harvested for RNA extraction using TriZOL (Thermo Fisher). Next, cDNA syntheses were performed using the SuperScript IV first-strand synthesis system, and quantitative RT-PCR using SYBR Green Dye with MTHFD2 specific primers was performed to assess relative MTHFD2 levels. Two previously studied MTHFD2 siRNAs were used as positive controls. Cumulative relative MTHFD2 expression based on six cell lines identified six siRNAs that were 30-50% more effective in inhibiting MTHFD2 levels.

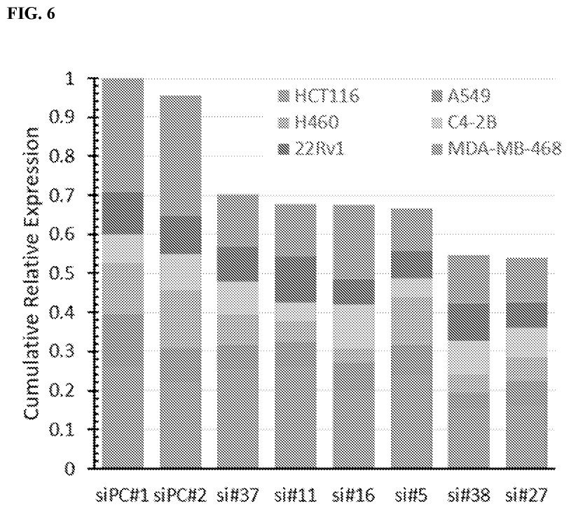

The data is presented in FIG. 6. The graph shows cumulative relative expression of MTHFD2 for six most potent siRNAs that were 30-50% more effective in inhibiting MTHFD2 levels compared to two MTHFD2 siRNAs that were used previously. Sequences of the most potent MTHFD2 siRNAs and two siRNAs that were used as the positive controls are presented below.

| Sequence | | |
|---|---|---|
| ID | Location | Sense Sequence |
| M_27 | 1007 | CGAGAAGUGCUGAAGUCUAAA |
| | | (SEQ ID NO: 99) |
| M_38 | 969 | CCAUUAUUGCUGCAAAAAAGG |
| | | (SEQ ID NO: 97) |
| M_05 | 292 | CAGUGAGACAAUUAUGAAACC |
| | | (SEQ ID NO: 69) |
| M_16 | 705 | CCAAAGAGCAGUUGAAGAAAC |
| | | (SEQ ID NO: 84) |
| M_11 | 406 | AGAGCAUAUUGAUGAGAGAAG |
| | | (SEQ ID NO: 77) |
| M_37 | 961 | GAAGAAUACCAUUAUUGCUGC |
| | | (SEQ ID NO: 96) |
| siPC#1 | 1722 | UAGGAUUAUUCCUUGCUAUUA |
| | | (SEQ ID NO: 141) |
| siPC#2 | 1140 | CAGGCCAGCUCAAGAAGCAAA |
| | | (SEQ ID NO: 142) |

Cell Viability Assays. Cells were reverse transfected with either non-targeting control or MTHFD2 targeting siRNAs using RNAiMAX reagent (Thermo Fisher) according to the manufacturer's protocols and plated into 96-well plates. 72 hours after transfection cell viability was determined using the Cell Count Kit-8 (CCK-8) assay. Briefly, 10 μL of WST-8 reagent solution containing 5 mM WST-8 and 0.2 mM 1-methoxy PMS in PBS, was added to each well, and plates were incubated at 37° C. in a humidified incubator with 5% CO2 for 2-4 hrs. After incubation, the absorbance was measured at 450 nm with a plate reader.

All publications, patents, patent applications and accession numbers mentioned in the above specification are herein incorporated by reference in their entirety. Although the invention has been described in connection with specific embodiments, it should be understood that the invention as claimed should not be unduly limited to such specific embodiments. Indeed, various modifications and variations of the described compositions and methods of the invention will be apparent to those of ordinary skill in the art and are intended to be within the scope of the following claims.

What is claimed is:

1. A double-stranded ribonucleic acid (dsRNA) agent for inhibiting expression of methylenetetrahydrofolate dehydrogenase 2 (MTHFD2), wherein said dsRNA agent comprises a sense strand and an antisense strand forming a double-stranded region, wherein said antisense strand comprises at least 15 contiguous nucleotides and excluding any overhang shares at least 80% identity to a sequence selected from the group consisting of SEQ ID NOs: 126 to 140.

2. The dsRNA agent of claim 1, wherein said dsRNA agent comprises at least one modified nucleotide.

3. The dsRNA agent of claim 2, wherein the at least one of said modified nucleotides is selected from the group consisting of a 2′-O-methyl modified nucleotide, a 2′-fluoro modified nucleotide, a 2′-deoxythymidine (dT) nucleotide, a nucleotide comprising a 5′-phosphorothioate group, and a terminal nucleotide linked to a cholesteryl derivative or a dodecanoic acid bisdecylamide group.

4. The dsRNA agent of any one of claims 1 to 3, wherein the antisense strand excluding any overhang differs by no more than 3 nucleotides from the sequence selected from the group consisting of SEQ ID NOs: 126 to 140.

5. The dsRNA agent of any one of claims 1 to 3, wherein the antisense strand excluding any overhang differs by no more than 2 nucleotides from the sequence selected from the group consisting of SEQ ID NOs: 126 to 140.

6. The dsRNA agent of any one of claims 1 to 5, wherein each strand is no more than 30 nucleotides in length.

7. The dsRNA agent of any one of claims 1 to 5, wherein each strand is independently 17-25 nucleotides in length.

8. The dsRNA agent of any one of claims 1 to 5, wherein each strand is independently 19-25 nucleotides in length.

9. The dsRNA agent of any one of claims 1 to 5, wherein each strand is independently 19-23 nucleotides in length.

10. The dsRNA agent of any one of claims 1 to 9, wherein at least one strand comprises a 3′ overhang of at least 1 nucleotide.

11. The dsRNA agent of any one of claims 1 to 9, wherein at least one strand comprises a 3′ overhang of at least 2 nucleotides.

12. The dsRNA agent of any one of claims 1 to 11, wherein said dsRNA agent further comprises at least one phosphorothioate or methylphosphonate internucleotide linkage.

13. A cell containing the dsRNA agent of any one of claims 1 to 12.

14. A pharmaceutical composition for inhibiting expression of a MTHFD2 gene comprising the dsRNA agent of any one of claims 1 to 12.

15. A method of inhibiting MTHFD2 expression in a cell, the method comprising: (a) contacting the cell with the dsRNA agent of any one of claims 1 to 12 or a pharmaceutical composition of claim 14; and (b) maintaining the cell produced in step (a) for a time sufficient to obtain degradation of the mRNA transcript of an gene, thereby inhibiting expression of the gene in the cell.

16. The method of claim 15, wherein said cell is within a subject.

17. A method of treating a subject having a disorder that would benefit from reduction in MTHFD2 expression, comprising administering to the subject a therapeutically effective amount of the dsRNA agent of any one of claims 1 to 12 or a pharmaceutical composition of claim 14, thereby treating said subject.

18. The method of claim 17, wherein the disorder is prostate cancer.

19. A method of inhibiting the expression of MTHFD2 in a subject, the method comprising administering to said subject a therapeutically effective amount of the dsRNA agent of any one f claims 1 to 12 or a pharmaceutical composition of claim 14, thereby inhibiting the expression of MTHFD2 in said subject.

20. dsRNA agent of any one of claims 1 to 12 or pharmaceutical composition of claim 14 for use in treating a subject having a disorder that would benefit from reduction in MTHFD2 expression.

21. dsRNA agent or pharmaceutical composition of claim 20, wherein the disorder is prostate cancer.

22. A method of inhibiting MTHFD2 expression in a subject in need thereof comprising:
administering to the subject a dsRNA agent comprising a sense strand and an antisense strand forming a double stranded region, wherein said antisense strand comprises at least 15 contiguous nucleotides and excluding any overhang is at least 80% identical to the nucleotide sequence of the complement of nucleotides 1 to 1100 of SEQ ID NO:64.

23. The method of claim 22, wherein said dsRNA agent comprises at least one modified nucleotide.

24. The method of claim 23, wherein the at least one of said modified nucleotides is selected from the group consisting of a 2′-O-methyl modified nucleotide, a 2′-fluoro modified nucleotide, a 2′-deoxythymidine (dT) nucleotide, a nucleotide comprising a 5′-phosphorothioate group, and a terminal nucleotide linked to a cholesteryl derivative or a dodecanoic acid bisdecylamide group.

25. The method agent of any one of claims 22 to 24, wherein each strand is no more than 30 nucleotides in length.

26. The method agent of any one of claims 22 to 24, wherein each strand is independently 17-25 nucleotides in length.

27. The method agent of any one of claims 22 to 24, wherein each strand is independently 19-25 nucleotides in length.

28. The method agent of any one of claims 22 to 24, wherein each strand is independently 19-23 nucleotides in length.

29. The method agent of any one of claims 22 to 24, wherein at least one strand comprises a 3′ overhang of at least 1 nucleotide.

30. The method agent of any one of claims 22 to 24, wherein at least one strand comprises a 3′ overhang of at least 2 nucleotides.

31. The method agent of any one of claims 22 to 30, wherein said dsRNA agent further comprises at least one phosphorothioate or methylphosphonate internucleotide linkage.

32. The method of any one of claims 22 to 31, wherein the dsRNA agent is formulated with a pharmaceutically acceptable carrier.

33. The method of any one of claims 22 to 32, wherein the subject has cancer.

34. The method of claim 33, wherein the cancer is prostate cancer.

35. An iRNA agent for inhibiting expression of methylenetetrahydrofolate dehydrogenase 2 (MTHFD2), wherein said iRNA agent comprises an antisense strand comprising at least 15 contiguous nucleotides and excluding any overhang shares at least 80% identity to a sequence selected from the group consisting of SEQ ID NOs: 126 to 140.

36. The iRNA agent of claim 35, wherein said iRNA agent comprises at least one modified nucleotide.

37. The iRNA agent of claim 36, wherein the at least one of said modified nucleotides is selected from the group consisting of a 2′-O-methyl modified nucleotide, a 2′-fluoro modified nucleotide, a 2′-deoxythymidine (dT) nucleotide, a nucleotide comprising a 5′-phosphorothioate group, and a terminal nucleotide linked to a cholesteryl derivative or a dodecanoic acid bisdecylamide group.

38. The iRNA agent of any one of claims 35 to 37, wherein the antisense strand excluding any overhang differs by no more than 3 nucleotides from the sequence selected from the group consisting of SEQ ID NOs: 126 to 140.

39. The iRNA agent of any one of claims 35 to 37, wherein the antisense strand excluding any overhang differs by no more than 2 nucleotides from the sequence selected from the group consisting of SEQ ID NOs: 126 to 140.

40. The iRNA agent of any one of claims 35 to 39, wherein the antisense strand is no more than 30 nucleotides in length.

41. The iRNA agent of any one of claims 35 to 39, wherein the antisense strand is independently 17-25 nucleotides in length.

42. The iRNA agent of any one of claims 35 to 39, wherein the antisense strand is independently 19-25 nucleotides in length.

43. The iRNA agent of any one of claims 35 to 39, wherein the antisense strand is independently 19-23 nucleotides in length.

44. The iRNA agent of any one of claims 35 to 43, wherein the antisense strand comprises a 3′ overhang of at least 1 nucleotide.

45. The iRNA agent of any one of claims 35 to 43, wherein the antisense strand comprises a 3′ overhang of at least 2 nucleotides.

46. The iRNA agent of any one of claims 35 to 45, wherein said iRNA agent further comprises at least one phosphorothioate or methylphosphonate internucleotide linkage.

47. The iRNA agent of any one of claims 35 to 46, wherein the iRNA agent is a dsRNA agent.

48. A cell containing the iRNA agent of any one of claims 35 to 47.

49. A pharmaceutical composition for inhibiting expression of a MTHFD2 gene comprising the iRNA agent of any one of claims 35 to 47.

50. A method of inhibiting MTHFD2 expression in a cell, the method comprising: (a) contacting the cell with the iRNA agent of any one of claims 35 to 47 or a pharmaceutical composition of claim 49; and (b) maintaining the cell produced in step (a) for a time sufficient to obtain degradation of the mRNA transcript of an gene, thereby inhibiting expression of the gene in the cell.

51. The method of claim 50, wherein said cell is within a subject.

52. A method of treating a subject having a disorder that would benefit from reduction in MTHFD2 expression, comprising administering to the subject a therapeutically effective amount of the iRNA agent of any one of claims 35 to 47 or a pharmaceutical composition of claim 49, thereby treating said subject.

53. The method of claim 52, wherein the disorder is prostate cancer.

54. A method of inhibiting the expression of MTHFD2 in a subject, the method comprising administering to said subject a therapeutically effective amount of the iRNA agent of any one of claims 35 to 47 or a pharmaceutical composition of claim 49, thereby inhibiting the expression of MTHFD2 in said subject.

55. iRNA agent of any one of claims 35 to 47 or pharmaceutical composition of claim 49 for use in treating a subject having a disorder that would benefit from reduction in MTHFD2 expression.

56. iRNA agent or pharmaceutical composition of claim 55, wherein the disorder is prostate cancer.

57. A method for treating prostate cancer, comprising administering to a subject in need thereof one or more oligonucleotides, or a salt thereof, or a pharmaceutical agent that induces the production of the one or more oligonucleotides, wherein the one or more oligonucleotides is at least 80% identical to any one of SEQ ID NOs: 1 to 63 or the complement thereof, wherein said oligonucleotides hybridize to the MTHFD2 of SEQ ID NO:64 or an mRNA encoded thereof.

58. The method of claim 57, wherein the one or more oligonucleotides is at least 90% identical to any one of SEQ ID NOs: 1 to 63 or the complement thereof.

59. The method of any one of claims 57 to 58, wherein the one or more nucleotides consist of from 15 to 40 linked nucleobases.

60. The method of any one of claims 56 to 58, wherein the one or more nucleotides is 100% identical to any of SEQ ID NOs: 1 to 63 or the complement thereof.

61. The method of any one of claims 57 to 60, wherein the one or more oligonucleotides is a modified oligonucleotide.

62. The method of any one of claims 57 to 61, wherein the one or more oligonucleotides is present in a pharmaceutical composition.

63. The method of any one of claims 57 to 62, wherein the one or more oligonucleotides is administered systemically or locally.

64. The method of claim 63, wherein the local administration is local administration to the prostate.

65. The method of any one of claims 57 to 64, wherein administration of the one or more oligonucleotides results in down-regulation of expression of the MTHFD2 gene in the tissue of a subject.

66. The method of any one of claims 57 to 65, wherein the subject has prostate cancer.

67. The method of claim 66, wherein the administration of the one or more nucleotides ameliorates one or more symptoms of prostate cancer in the subject.

68. The method of any one of claims 56 to 66, wherein the oligonucleotide is an RNA.

69. The method of claim 68, wherein said RNA is an siRNA.

70. The method of claim 69, wherein said siRNA is a double stranded RNA comprising a sense strand and an antisense strand, wherein said antisense strand hybridizes to an mRNA encoded by SEQ ID NO:64.

71. Oligonucleotide composition comprising one or more nucleotides, or salts thereof, or a pharmaceutical agent that induces the production of the one or more oligonucleotide, wherein the one or more oligonucleotides is at least 80% identical to any one of SEQ ID NOs: 1 to 63 or the complement thereof, wherein said oligonucleotides hybridize to the MTHFD2 of SEQ ID NO:64 or an mRNA encoded thereof.

72. Oligonucleotide composition of claim 71, wherein the one or more oligonucleotides is at least 90% identical to any one of SEQ ID NOs: 1 to 63 or the complement thereof.

73. Oligonucleotide composition of any one of claims 71 to 72, wherein the one or more nucleotides consist of from 15 to 40 linked nucleobases.

74. Oligonucleotide composition of any one of claims 71 to 73, wherein the one or more nucleotides consist of any one of SEQ ID NOs: 1 to 63 or the complement thereof.

75. Oligonucleotide composition of any one of claims 71 to 74, wherein the one or more oligonucleotides is a modified oligonucleotide.

76. Oligonucleotide composition of any one of claims 71 to 75, wherein the one or more oligonucleotides is present in a pharmaceutical composition.

77. Oligonucleotide composition of any one of claims 71 to 76, wherein the one or more oligonucleotides is administered systemically or locally.

78. Oligonucleotide composition of claim 77, wherein the local administration is local administration to the prostate.

79. Oligonucleotide composition of any one of claims 71 to 78, wherein administration of the one or more oligonucleotides results in down-regulation of expression of the MTHFD2 gene in the tissue of a subject.

80. Oligonucleotide composition of any one of claims 71 to 79, wherein the subject has prostate cancer.

81. Oligonucleotide composition of claim 80, wherein the administration of the one or more nucleotides ameliorates one or more symptoms of prostate cancer in the subject.

82. Oligonucleotide composition of any one of claims 71 to 81, wherein the oligonucleotide is an RNA.

83. Oligonucleotide composition of claim 82, wherein the oligonucleotide is an siRNA.

84. Oligonucleotide composition of claim 83, wherein said siRNA is a double stranded RNA comprising a sense strand and an antisense strand, wherein said antisense strand hybridizes to an mRNA expressed by SEQ ID NO:64.

85. Oligonucleotide composition of any one of claims 71 to 84 for use in treating prostate cancer in a subject.

86. An expression vector encoding an siRNA comprising a nucleic acid that expresses an RNA selected from the group consisting of SEQ ID NOs: 1-63 linked to a nucleic acid encoding the complement of an RNA selected from the group consisting of SEQ ID NOs: 1-63.

87. A method for treating prostate cancer, comprising administering to a subject in need thereof the expression vector of claim 86.

88. Expression vector of claim 86 for use in treating prostate cancer in a subject.


SEQUENCE LISTING (142 sequences)

SEQ ID NO: 1 - RNA, 21 nt, Artificial sequence
  synthetic
uucauuucgaacugccgcgag

SEQ ID NO: 2 - RNA, 21 nt, Artificial sequence
  synthetic
uuccagaaaugacaacagcuu

SEQ ID NO: 3 - RNA, 21 nt, Artificial sequence
  synthetic
ugacuugcaggauucucgcca

SEQ ID NO: 4 - RNA, 21 nt, Artificial sequence
  synthetic
uugaggacauaggagugacuu

SEQ ID NO: 5 - RNA, 21 nt, Artificial sequence
  synthetic
uuucauaauugucucacuguu

SEQ ID NO: 6 - RNA, 21 nt, Artificial sequence
  synthetic
uuccucugaaauugaagcugg

SEQ ID NO: 7 - RNA, 21 nt, Artificial sequence
  synthetic
aauucuuccucugaaauugaa

SEQ ID NO: 8 - RNA, 21 nt, Artificial sequence
  synthetic
uucaacaauucuuccucugaa

SEQ ID NO: 9 - RNA, 21 nt, Artificial sequence
  synthetic
uuaaauucaacaauucuuccu

SEQ ID NO: 10 - RNA, 21 nt, Artificial sequence
  synthetic
uuauugauuaaauucaacaau

SEQ ID NO: 11 - RNA, 21 nt, Artificial sequence
  synthetic
aucauuauucaguuuauugau

SEQ ID NO: 12 - RNA, 21 nt, Artificial sequence
  synthetic
uacauuaucaucauuauucag

SEQ ID NO: 13 - RNA, 21 nt, Artificial sequence
  synthetic
ucucucaucaauaugcucugg

SEQ ID NO: 14 - RNA, 21 nt, Artificial sequence
  synthetic
uugucuggagaaacagcauug

SEQ ID NO: 15 - RNA, 21 nt, Artificial sequence
  synthetic
acauuaauuacaugaaagcca

SEQ ID NO: 16 - RNA, 21 nt, Artificial sequence
  synthetic
uacugauccaaacacauucgu

SEQ ID NO: 17 - RNA, 21 nt, Artificial sequence
  synthetic
uaacauggaauacugauccaa

SEQ ID NO: 18 - RNA, 21 nt, Artificial sequence
  synthetic
augagauauuguaacaguggc

SEQ ID NO: 19 - RNA, 21 nt, Artificial sequence
  synthetic
uauaucgaugagauauuguaa

SEQ ID NO: 20 - RNA, 21 nt, Artificial sequence
  synthetic
uucuucaacugcucuuuggga

SEQ ID NO: 21 - RNA, 21 nt, Artificial sequence
  synthetic
uauguuucuucaacugcucuu

SEQ ID NO: 22 - RNA, 21 nt, Artificial sequence
  synthetic
aagaauuguauguuucuucaa

SEQ ID NO: 23 - RNA, 21 nt, Artificial sequence
  synthetic
uaucugcaagaauuguauguu

SEQ ID NO: 24 - RNA, 21 nt, Artificial sequence
  synthetic
uacaauaucugcaagaauugu

SEQ ID NO: 25 - RNA, 21 nt, Artificial sequence
  synthetic
ucagauuuggaauaccugcag

SEQ ID NO: 26 - RNA, 21 nt, Artificial sequence
  synthetic
uuccuugaucauaucugcugu

SEQ ID NO: 27 - RNA, 21 nt, Artificial sequence
  synthetic
auuuauucccacaucaaugac

SEQ ID NO: 28 - RNA, 21 nt, Artificial sequence
  synthetic
uuacaggaucgugaacucuau

SEQ ID NO: 29 - RNA, 21 nt, Artificial sequence
  synthetic
uccacaucuccaaccaacuug

SEQ ID NO: 30 - RNA, 21 nt, Artificial sequence
  synthetic
ugauauacccagcuuuuuguc

SEQ ID NO: 31 - RNA, 21 nt, Artificial sequence
  synthetic
uauucuucauuagcauugcca

SEQ ID NO: 32 - RNA, 21 nt, Artificial sequence
  synthetic
agcaauaaugguauucuucau

SEQ ID NO: 33 - RNA, 21 nt, Artificial sequence
  synthetic
uuuuuugcagcaauaauggua

SEQ ID NO: 34 - RNA, 21 nt, Artificial sequence
  synthetic
uucucgcucuucaagccucag

SEQ ID NO: 35 - RNA, 21 nt, Artificial sequence
  synthetic
uagacuucagcacuucucgcu

SEQ ID NO: 36 - RNA, 21 nt, Artificial sequence
  synthetic
uuugugacacagaagacacag

SEQ ID NO: 37 - RNA, 21 nt, Artificial sequence
  synthetic
aauaaauuaaaaauauugcau

SEQ ID NO: 38 - RNA, 21 nt, Artificial sequence
  synthetic
uuaagcuuucaauaaauacaa

SEQ ID NO: 39 - RNA, 21 nt, Artificial sequence
  synthetic
uaaucacuagguuaauggcug

SEQ ID NO: 40 - RNA, 21 nt, Artificial sequence
  synthetic
uugacaaagugaagcauccau

SEQ ID NO: 41 - RNA, 21 nt, Artificial sequence
  synthetic
uauugcaauagcacuugggaa

SEQ ID NO: 42 - RNA, 21 nt, Artificial sequence
  synthetic
uucaauaguaacucaaacucu

SEQ ID NO: 43 - RNA, 21 nt, Artificial sequence
  synthetic
uuaaauucaauaguaacucaa

SEQ ID NO: 44 - RNA, 21 nt, Artificial sequence
  synthetic
uaggucacaacuaggagucau

SEQ ID NO: 45 - RNA, 21 nt, Artificial sequence
  synthetic
uauaggaauagcuacauugau

SEQ ID NO: 46 - RNA, 21 nt, Artificial sequence
  synthetic
uaagacuguaggacaggccag

SEQ ID NO: 47 - RNA, 21 nt, Artificial sequence
  synthetic
uucucuuucugccucaagcug

SEQ ID NO: 48 - RNA, 21 nt, Artificial sequence
  synthetic
uuuaauaaucuagguaagcag

SEQ ID NO: 49 - RNA, 21 nt, Artificial sequence
  synthetic
uaauccugucuggucaucuug

SEQ ID NO: 50 - RNA, 21 nt, Artificial sequence
  synthetic
uaacagguugacauaauacuu

SEQ ID NO: 51 - RNA, 21 nt, Artificial sequence
  synthetic
uuacaugucauuuauauuuug

SEQ ID NO: 52 - RNA, 21 nt, Artificial sequence
  synthetic
uuguuguaaauugaucacugu

SEQ ID NO: 53 - RNA, 21 nt, Artificial sequence
  synthetic
uugauuaaucaaacaagcugg

SEQ ID NO: 54 - RNA, 21 nt, Artificial sequence
  synthetic
uuaucauccacaccaauccug

SEQ ID NO: 55 - RNA, 21 nt, Artificial sequence
  synthetic
uuaaguauaaauucuugucag

SEQ ID NO: 56 - RNA, 21 nt, Artificial sequence
  synthetic
uuugauuacauuuaaucucag

SEQ ID NO: 57 - RNA, 21 nt, Artificial sequence
  synthetic
ugaguaaaucacuugaaccuu

SEQ ID NO: 58 - RNA, 21 nt, Artificial sequence
  synthetic
uuaaugccuacuaugacccug

SEQ ID NO: 59 - RNA, 21 nt, Artificial sequence
  synthetic
uuaaugaacaccaaccauguu

SEQ ID NO: 60 - RNA, 21 nt, Artificial sequence
  synthetic
uuugcaauauuaaucaaccua

SEQ ID NO: 61 - RNA, 21 nt, Artificial sequence
  synthetic
uucaugugaacaauacagguu

SEQ ID NO: 62 - DNA, 21 bp, Artificial sequence
  synthetic
taggattattccttgctatta

SEQ ID NO: 63 - DNA, 21 bp, Artificial sequence
  synthetic
caggccagctcaagaagcaaa

SEQ ID NO: 64 - DNA, 4403 bp, Artificial sequence
  synthetic
ctcccggcgcagtcaccggcgcggtctatggctgcgacttctctaatgtctgctttggctgcccggctgctgcagcccgcgcacagctgctcccttcgccttcgccctttccacctcgcggcagttcgaaatgaagctgttgtcatttctggaaggaaactggcccagcagatcaagcaggaagtgcggcaggaggtagaagagtgggtggcctcaggcaacaaacggccacacctgagtgtgatcctggttggcgagaatcctgcaagtcactcctatgtcctcaacaaaaccagggcagctgcagttgtgggaatcaacagtgagacaattatgaaaccagcttcaatttcagaggaagaattgttgaatttaatcaataaactgaataatgatgataatgtagatggcctccttgttcagttgcctcttccagagcatattgatgagagaaggatctgcaatgctgtttctccagacaaggatgttgatggctttcatgtaattaatgtaggacgaatgtgtttggatcagtattccatgttaccggctactccatggggtgtgtgggaaataatcaagcgaactggcattccaaccctagggaagaatgtggttgtggctggaaggtcaaaaaacgttggaatgcccattgcaatgttactgcacacagatggggcgcatgaacgtcccggaggtgatgccactgttacaatatctcatcgatatactcccaaagagcagttgaagaaacatacaattcttgcagatattgtaatatctgctgcaggtattccaaatctgatcacagcagatatgatcaaggaaggagcagcagtcattgatgtgggaataaatagagttcacgatcctgtaactgccaaacccaagttggttggagatgtggattttgaaggagtcagacaaaaagctgggtatatcactccagttcctggaggtgttggccccatgacagtggcaatgctaatgaagaataccattattgctgcaaaaaaggtgctgaggcttgaagagcgagaagtgctgaagtctaaagagcttggggtagccactaattaactactgtgtcttctgtgtcacaaacagcactccaggccagctcaagaagcaaagcaggccaatagaaatgcaatatttttaatttattctactgaaatggtttaaaatgatgccttgtatttattgaaagcttaaatgggtgggtgtttctgcacatacctctgcagtacctcaccagggagcattccagtatcatgcagggtcctgtgatctagccaggagcagccattaacctagtgattaatatgggagacattaccatatggaggatggatgcttcactttgtcaagcacctcagttacacattcgccttttctaggattgcatttcccaagtgctattgcaataacagttgatactcattttaggtaccaaaccttttgagttcaactgatcaaaccaaaggaaaagtgttgctagagaaaattagggaaaaggtgaaaaagaaaaaatggtagtaattgagcagaaaaaaattaatttatatatgtattgattggcaaccagatttatctaagtagaactgaattggctaggaaaaaagaaaaactgcatgttaatcattttcctaagctgtccttttgaggcttagtcagtttattgggaaaatgtttaggattattccttgctattagtactcattttatgtatgttacccttcagtaagttctccccattttagttttctaggactgaaaggattcttttctacattatacatgtgtgttgtcatatttggcttttgctatatactttaacttcattgttaaatttttgtattgtatagtttctttggtgtatcttaaaacctatttttgaaaaacaaacttggcttgataatcatttgggcagcttgggtaagtacgcaacttacttttccaccaaagaactgtcagcagctgcctgcttttctgtgatgtatgtatcctgttgacttttccagaaattttttaagagtttgagttactattgaatttaatcagactttctgattaaagggttttctttcttttttaataaaacacatctgtctggtatggtatgaatttctgaaattctgtcttcctatgactcctagttgtgacctaaccagtttttttctcatttaatcaatgtagctattcctatattcagtaacacttacttctatagccttaaatagataattttttttcttctatttttttttttttttttgtagagatggggtcttgctatgttgcctgggctggtattgaactcctggcctcaagcaatcaatcctcttgcctcagcttcccagagggctgggattacaggcctgggccacccacacccaaacagaggtttaaagagctttggagaaaccagcctcgtcctccagtctctttgtttgtttgttttgagactgagtcttgctctgttgcccaggctggagtgcagtggcatgatctcgggtcactgcaacctctgcctctcgggttcaaacaattcttgtgcctcagcctcctgagtagctgggattacaggtgcatgccaccatgcctggctaatttttgtgtttttagtagagatggggtttcatcacattggccaggctggtctcaaactcctgacctcaagtgatccacctgcctcggcctcccaaaatgctaggattacaggcgtgagccactgcacctggcctgtcctacagtcttatatcacaacaggttgtgccaagctaggagcttaatctgtaaaatacttgaggcagggataggtttgtctgttttggcttttggctttcatgcgagcagaagtagctcctgctctaggcagcttgaggcagaaagagaagaaacaattgagttgacctacctccgtcctcgttcttctgccagctggcgttattcttttgatagttctctggagtttaaagaagtttagatagtttgctggagatgtttttgtaagtgacgtatagcttatgtctctaatccatgcctgcttacctagattattaaatgtggagcaagatgaccagacaggattaaattttattgaattacagttacaagtggaaaaaatgactgacttactgcatggagaagtattatgtcaacctgttaaaagctatggaaattaaaatgttcatgtatatacaaaatataaatgacatgtaaggtaaaattacagtgatcaatttacaacaaagaaattaacagtagttttccagcttgtttgattaatcaagcaggtttactcttgaatccatagcatttctacaatttaatttggacaaagttctattccagatcagccaggttttaaaactgaaatgtgttaagggtggattgttagcttccccaggattggtgtggatgataaatactttttttaaaaattaattattattgtttataagacatggattccagctgtctccctggctggagtgcagtggcatgattatagctcactgcagccttgaactcctggacttaaacgatcctcctgcatcagcctctgaaatggctgggactacaggtgtgtgccagcatgcctagctaatttttaaacatttgtttagagatgggtctcactatgttgcccaggctggtcttgacctcctgacctctgctgattccccccacctctgcctcccaagtagcgggattacaggtgtgagccactgtgtctgacaagaatttatacttaagcataggagatggttctggaaattctaagaaattctgctctcagtaagagtagaggtttggagctttacctcttggcagtatcccttggaagggagctctgagattaaatgtaatcaaacacttattgagagctttctatggaccaggcactacactagacgctgggaattacagaggcaattacctgtcctatactcaaggtgttaatgatctagtgtcatttttctttaaaaaagaaagcacctgcatttgtcatcttgagcaaatatagtaactctaacctcaaggttcaagtgatttactcaaggtttaaatcatagttgttttggagaaagtaagtgtcagatgcactgtggctgtcagggtcatagtaggcattaaatcaacatggttggtgttcattaaatgtggattgaagataaaatgactgatcttaagtttcatgaggaataggtttgcttcattgaaactggtttttatcttcccaaatagttttcaatcattcctttaaagtactaggttgattaatattgcaaagttgtaatgtaaaatgtgaccttacctctgtctaacctgtattgttcacatgaatgggagtttgactttgggaataaacattgaaaatttgcagggaggaccca

SEQ ID NO: 65 - RNA, 21 nt, Artificial sequence
  synthetic
cgcggcaguucgaaaugaagc

SEQ ID NO: 66 - RNA, 21 nt, Artificial sequence
  synthetic
gcuguugucauuucuggaagg

SEQ ID NO: 67 - RNA, 21 nt, Artificial sequence
  synthetic
gcgagaauccugcaagucacu

SEQ ID NO: 68 - RNA, 21 nt, Artificial sequence
  synthetic
gucacuccuauguccucaaca

SEQ ID NO: 69 - RNA, 21 nt, Artificial sequence
  synthetic
cagugagacaauuaugaaacc

SEQ ID NO: 70 - RNA, 21 nt, Artificial sequence
  synthetic
agcuucaauuucagaggaaga

SEQ ID NO: 71 - RNA, 21 nt, Artificial sequence
  synthetic
caauuucagaggaagaauugu

SEQ ID NO: 72 - RNA, 21 nt, Artificial sequence
  synthetic
cagaggaagaauuguugaauu

SEQ ID NO: 73 - RNA, 21 nt, Artificial sequence
  synthetic
gaagaauuguugaauuuaauc

SEQ ID NO: 74 - RNA, 21 nt, Artificial sequence
  synthetic
uguugaauuuaaucaauaaac

SEQ ID NO: 75 - RNA, 21 nt, Artificial sequence
  synthetic
caauaaacugaauaaugauga

SEQ ID NO: 76 - RNA, 21 nt, Artificial sequence
  synthetic
gaauaaugaugauaauguaga

SEQ ID NO: 77 - RNA, 21 nt, Artificial sequence
  synthetic
agagcauauugaugagagaag

SEQ ID NO: 78 - RNA, 21 nt, Artificial sequence
  synthetic
augcuguuucuccagacaagg

SEQ ID NO: 79 - RNA, 21 nt, Artificial sequence
  synthetic
gcuuucauguaauuaauguag

SEQ ID NO: 80 - RNA, 21 nt, Artificial sequence
  synthetic
gaauguguuuggaucaguauu

SEQ ID NO: 81 - RNA, 21 nt, Artificial sequence
  synthetic
ggaucaguauuccauguuacc

SEQ ID NO: 82 - RNA, 21 nt, Artificial sequence
  synthetic
cacuguuacaauaucucaucg

SEQ ID NO: 83 - RNA, 21 nt, Artificial sequence
  synthetic
acaauaucucaucgauauacu

SEQ ID NO: 84 - RNA, 21 nt, Artificial sequence
  synthetic
ccaaagagcaguugaagaaac

SEQ ID NO: 85 - RNA, 21 nt, Artificial sequence
  synthetic
gagcaguugaagaaacauaca

SEQ ID NO: 86 - RNA, 21 nt, Artificial sequence
  synthetic
gaagaaacauacaauucuugc

SEQ ID NO: 87 - RNA, 21 nt, Artificial sequence
  synthetic
cauacaauucuugcagauauu

SEQ ID NO: 88 - RNA, 21 nt, Artificial sequence
  synthetic
aauucuugcagauauuguaau

SEQ ID NO: 89 - RNA, 21 nt, Artificial sequence
  synthetic
gcagguauuccaaaucugauc

SEQ ID NO: 90 - RNA, 21 nt, Artificial sequence
  synthetic
agcagauaugaucaaggaagg

SEQ ID NO: 91 - RNA, 21 nt, Artificial sequence
  synthetic
cauugaugugggaauaaauag

SEQ ID NO: 92 - RNA, 21 nt, Artificial sequence
  synthetic
agaguucacgauccuguaacu

SEQ ID NO: 93 - RNA, 21 nt, Artificial sequence
  synthetic
aguugguuggagauguggauu

SEQ ID NO: 94 - RNA, 21 nt, Artificial sequence
  synthetic
caaaaagcuggguauaucacu

SEQ ID NO: 95 - RNA, 21 nt, Artificial sequence
  synthetic
gcaaugcuaaugaagaauacc

SEQ ID NO: 96 - RNA, 21 nt, Artificial sequence
  synthetic
gaagaauaccauuauugcugc

SEQ ID NO: 97 - RNA, 21 nt, Artificial sequence
  synthetic
ccauuauugcugcaaaaaagg

SEQ ID NO: 98 - RNA, 21 nt, Artificial sequence
  synthetic
gaggcuugaagagcgagaagu

SEQ ID NO: 99 - RNA, 21 nt, Artificial sequence
  synthetic
cgagaagugcugaagucuaaa

SEQ ID NO: 100 - RNA, 21 nt, Artificial sequence
  synthetic
gugucuucugugucacaaaca

SEQ ID NO: 101 - RNA, 21 nt, Artificial sequence
  synthetic
gcaauauuuuuaauuuauucu

SEQ ID NO: 102 - RNA, 21 nt, Artificial sequence
  synthetic
guauuuauugaaagcuuaaau

SEQ ID NO: 103 - RNA, 21 nt, Artificial sequence
  synthetic
gccauuaaccuagugauuaau

SEQ ID NO: 104 - RNA, 21 nt, Artificial sequence
  synthetic
ggaugcuucacuuugucaagc

SEQ ID NO: 105 - RNA, 21 nt, Artificial sequence
  synthetic
cccaagugcuauugcaauaac

SEQ ID NO: 106 - RNA, 21 nt, Artificial sequence
  synthetic
aguuugaguuacuauugaauu

SEQ ID NO: 107 - RNA, 21 nt, Artificial sequence
  synthetic
gaguuacuauugaauuuaauc

SEQ ID NO: 108 - RNA, 21 nt, Artificial sequence
  synthetic
gacuccuaguugugaccuaac

SEQ ID NO: 109 - RNA, 21 nt, Artificial sequence
  synthetic
caauguagcuauuccuauauu

SEQ ID NO: 110 - RNA, 21 nt, Artificial sequence
  synthetic
ggccuguccuacagucuuaua

SEQ ID NO: 111 - RNA, 21 nt, Artificial sequence
  synthetic
gcuugaggcagaaagagaaga

SEQ ID NO: 112 - RNA, 21 nt, Artificial sequence
  synthetic
gcuuaccuagauuauuaaaug

SEQ ID NO: 113 - RNA, 21 nt, Artificial sequence
  synthetic
agaugaccagacaggauuaaa

SEQ ID NO: 114 - RNA, 21 nt, Artificial sequence
  synthetic
guauuaugucaaccuguuaaa

SEQ ID NO: 115 - RNA, 21 nt, Artificial sequence
  synthetic
aaauauaaaugacauguaagg

SEQ ID NO: 116 - RNA, 21 nt, Artificial sequence
  synthetic
agugaucaauuuacaacaaag

SEQ ID NO: 117 - RNA, 21 nt, Artificial sequence
  synthetic
agcuuguuugauuaaucaagc

SEQ ID NO: 118 - RNA, 21 nt, Artificial sequence
  synthetic
ggauugguguggaugauaaau

SEQ ID NO: 119 - RNA, 21 nt, Artificial sequence
  synthetic
gacaagaauuuauacuuaagc

SEQ ID NO: 120 - RNA, 21 nt, Artificial sequence
  synthetic
gagauuaaauguaaucaaaca

SEQ ID NO: 121 - RNA, 21 nt, Artificial sequence
  synthetic
gguucaagugauuuacucaag

SEQ ID NO: 122 - RNA, 21 nt, Artificial sequence
  synthetic
gggucauaguaggcauuaaau

SEQ ID NO: 123 - RNA, 21 nt, Artificial sequence
  synthetic
caugguugguguucauuaaau

SEQ ID NO: 124 - RNA, 21 nt, Artificial sequence
  synthetic
gguugauuaauauugcaaagu

SEQ ID NO: 125 - RNA, 21 nt, Artificial sequence
  synthetic
ccuguauuguucacaugaaug

SEQ ID NO: 126 - RNA, 19 nt, Artificial sequence
  synthetic
uagacuucagcacuucucg

SEQ ID NO: 127 - RNA, 19 nt, Artificial sequence
  synthetic
uuuuuugcagcaauaaugg

SEQ ID NO: 128 - RNA, 19 nt, Artificial sequence
  synthetic
uuucauaauugucucacug

SEQ ID NO: 129 - RNA, 19 nt, Artificial sequence
  synthetic
uucuucaacugcucuuugg

SEQ ID NO: 130 - RNA, 19 nt, Artificial sequence
  synthetic
ucucucaucaauaugcucu

SEQ ID NO: 131 - RNA, 19 nt, Artificial sequence
  synthetic
agcaauaaugguauucuuc

SEQ ID NO: 132 - RNA, 19 nt, Artificial sequence
  synthetic
uucaacaauucuuccucug

SEQ ID NO: 133 - RNA, 19 nt, Artificial sequence
  synthetic
uuaaauucaacaauucuuc

SEQ ID NO: 134 - RNA, 19 nt, Artificial sequence
  synthetic
uaacauggaauacugaucc

SEQ ID NO: 135 - RNA, 19 nt, Artificial sequence
  synthetic
ucagauuuggaauaccugc

SEQ ID NO: 136 - RNA, 19 nt, Artificial sequence
  synthetic
uccacaucuccaaccaacu

SEQ ID NO: 137 - RNA, 19 nt, Artificial sequence
  synthetic
ugauauacccagcuuuuug

SEQ ID NO: 138 - RNA, 19 nt, Artificial sequence
  synthetic
uuaagcuuucaauaaauac

SEQ ID NO: 139 - RNA, 19 nt, Artificial sequence
  synthetic
uaaucacuagguuaauggc

SEQ ID NO: 140 - RNA, 19 nt, Artificial sequence
  synthetic
uauugcaauagcacuuggg

SEQ ID NO: 141 - RNA, 21 nt, Artificial sequence
  synthetic
uaggauuauuccuugcuauua

SEQ ID NO: 142 - RNA, 21 nt, Artificial sequence
  synthetic
caggccagcucaagaagcaaa